(19) United States
(12) Patent Application Publication
(10) Pub. No.: US 2026/0347287 A1
(43) Pub. Date: Nov. 5, 2026

(54) NAPHTHA PRODUCTION FROM RECYCLED CONTENT PYROLYSIS OIL AS FEEDSTOCK TO FLUIDIZED CATALYTIC CRACKER

(71) Applicant: Eastman Chemical Company, Kingsport, TN (US)

(72) Inventors: Xianchun Wu, Longview, TX (US); Daryl Bitting, Longview, TX (US); Nick Allen Collins, Fall Branch, TN (US); Michael Gary Polasek, Richmond, TX (US); David Eugene Slivensky, Tatum, TX (US)

(73) Assignee: Eastman Chemical Company, Kingsport, TN (US)

(21) Appl. No.: 19/100,199

(22) PCT Filed: Jul. 20, 2023
(86) PCT No.: PCT/US2023/070575

§ 371 Date: Jan. 31, 2025

Related U.S. Application Data
(60) Provisional application No. 63/370,252, filed on Aug. 3, 2022.

(51) Int. Cl. C07C 6/12; C07C 15/08; C07C 51/265; C07C 63/26; C10G 69/10

(52) U.S. Cl. CPC C07C 6/126; C07C 15/08; C07C 51/265; C07C 63/26; C10G 69/10; C10G 2300/1007; C10G 2400/30

(57) ABSTRACT

Processes and facilities for producing a recycled content organic chemical compound directly or indirectly from waste plastic. Processing schemes are described herein for converting waste plastic (or hydrocarbon having recycled content derived from waste plastic) into useful intermediate chemicals and final products. In some aspects, recycled content aromatics (r-aromatics) can be processed to provide recycled content paraxylene (r-paraxylene), which can then be used to provide recycled content terephthalic acid (r-TPA) and/or recycled content polyethylene terephthalate (r-PET).

### BACKGROUND

Aromatic compounds such as benzene, toluene, and xylenes are important industrial chemicals used in a variety of applications. Paraxylene is used to form dicarboxylic acids and esters, which are key chemical feedstocks in the production of polyesters and aromatics-based plasticizers. Most conventional production routes for these materials utilize fossil fuel-derived feeds. Thus, it would be desirable to find additional synthesis routes for paraxylene and other aromatics that are sustainable, while also providing high-purity end products. Advantageously, the manufacture of such components can be carried out with existing equipment and facilities.

### SUMMARY

In one aspect, the present technology concerns a method for producing recycled content paraxylene (r-pX), the method comprising: (a) distilling recycled content pyrolysis oil (r-pyoil) and crude oil in at least one distillation column to produce at least one recycled content heavy hydrocarbon (r-heavy hydrocarbon) stream having a median boiling point (T50) of greater than 400 °F; (b) reducing an average molecular weight of at least a portion of the r-heavy hydrocarbon stream to produce at least one recycled content naphtha (r-naphtha) stream, wherein the r-naphtha stream comprises at least one recycled content aromatics (r-aromatics) stream and/or wherein at least a portion of the r-naphtha stream is further processed to produce an r-aromatics stream; and (c) processing at least a portion of the r-aromatics stream within an aromatics complex to produce a recycled content paraxylene (r-paraxylene) stream comprising at least 85 weight percent paraxylene.

In one aspect, the present technology concerns a method for producing recycled content paraxylene (r-paraxylene), the method comprising: (a) distilling recycled content pyrolysis oil (r-pyoil) and crude oil in an atmospheric distillation column to produce at least one recycled content heavy hydrocarbon (r-heavy hydrocarbon) stream having a median boiling point (T50) of greater than 650 °F; (b) separating at least a portion of the r-heavy hydrocarbon stream in a vacuum distillation unit (VDU) to produce at least a recycled content light vacuum gas oil (r-LVGO) stream having a T50 of greater than 650 °F and not more than 800 °F; (c) catalytically cracking at least a portion of the r-LVGO stream in a fluidized catalytic cracker (FCC) unit to produce at least one recycled content naphtha (r-naphtha) stream, wherein the r-naphtha stream comprises at least one recycled content aromatics (r-aromatics) stream and/or wherein at least a portion of the r-naphtha stream is further processed to produce at least one recycled content aromatics (r-aromatics) stream; and (d) processing at least a portion of the r-aromatics stream within an aromatics complex to produce an recycled content paraxylene (r-paraxylene) stream comprising at least 85 weight percent paraxylene.

In one aspect, the present technology concerns a method for producing recycled content paraxylene (r-paraxylene), the method comprising: (a) distilling recycled content pyrolysis oil (r-pyoil) and crude oil in an atmospheric distillation column to produce a recycled content atmospheric gas oil (r-AGO) stream having a median boiling point (T50) of greater than 400 °F and not more than 650 °F; (b) catalytically cracking at least a portion of the r-AGO stream in an FCC unit to produce at least one recycled content naphtha (r-naphtha) stream, wherein the r-naphtha stream comprises at least one recycled content aromatics (r-aromatics) stream and/or wherein at least a portion of the r-naphtha stream is further processed to produce at least one r-aromatics stream; and (c) processing at least a portion of the r-aromatics stream within an aromatics complex to produce a recycled content paraxylene (r-paraxylene) stream comprising at least 85 weight percent paraxylene.

In one aspect, the present technology concerns a method for producing recycled content paraxylene (r-pX), the method comprising: (a) distilling recycled content pyrolysis oil (r-pyoil) and crude oil in an atmospheric distillation column to produce at least one recycled content heavy hydrocarbon (r-heavy hydrocarbon) stream having a median boiling point (T50) of greater than 650 °F and a recycled content atmospheric gas oil (r-AGO) stream having a T50 of greater than 400 °F and not more than 650 °F; (b) separating at least a portion of the r-heavy hydrocarbon stream in a vacuum distillation unit (VDU) to produce at least a recycled content light vacuum gas oil (r-LVGO) stream having a T50 of greater than 650 °F and not more than 800 °F; (c) catalytically cracking at least a portion of the r-AGO stream and at least a portion of the r-LVGO stream in a fluidized catalytic cracking (FCC) unit to produce at least one recycled content naphtha (r-naphtha) stream, wherein the r-naphtha stream comprises at least one recycled content aromatics (r-aromatics) stream and/or wherein at least a portion of the r-naphtha stream is further processed to produce at least one r-aromatics stream; and (d) processing at least a portion of the r-aromatics stream within an aromatics complex to produce a recycled content paraxylene (r-paraxylene) stream comprising at least 85 weight percent paraxylene.

In one aspect, the present technology concerns a method for producing a recycled content organic chemical compound (r-organic chemical compound), the method comprising: (a) introducing a recycled content aromatics (r-aromatics) stream into an aromatics complex, wherein the r-aromatics stream is obtained by distilling recycled content pyrolysis oil (r-pyoil) and crude oil in at least one distillation column of a refinery to produce at least one recycled content heavy hydrocarbon (r-heavy hydrocarbon) stream having a median boiling point (T50) of greater than 400 °F, reducing an average molecular weight of the r-heavy hydrocarbon stream to produce at least one recycled content naphtha (r-naphtha) stream, wherein the r-naphtha stream comprises the r-aromatics stream and/or wherein at least a portion of the r-naphtha stream is further processed to produce the r-aromatics stream; and (b) processing at least a portion of the r-aromatics stream in the aromatics complex to provide a recycled content aromatics (r-aromatics) stream comprising at least 85 weight percent paraxylene.

In one aspect, the present technology concerns a method for producing a recycled content organic chemical compound (r-organic chemical compound), the method comprising: (a) introducing a stream of recycled content paraxylene (r-paraxylene) into a terephthalic acid (TPA) production facility, wherein at least a portion of the r-paraxylene is obtained by distilling recycled content pyrolysis oil (r-pyoil) and crude oil in at least one distillation column of a refinery to produce at least one recycled content heavy hydrocarbon (r-heavy hydrocarbon) stream having a median boiling point (T50) of greater than 400 °F, reducing an average molecular weight of at least a portion of the r-heavy hydrocarbon stream to produce at least one recycled content naphtha (r-naphtha) stream, wherein the r-naphtha stream comprises a recycled content aromatics (r-aromatics) stream and/or wherein at least a portion of the r-naphtha stream is further processed to produce the r-aromatics stream, and processing the r-aromatics stream in an aromatics complex to provide the r-paraxylene stream; and (b) processing at least a portion of the r-paraxylene stream in the TPA production facility to provide recycled content purified terephthalic acid (r-PTA).

### BRIEF DESCRIPTION OF THE DRAWINGS

FIG. 1a is a block flow diagram illustrating the main steps of a process for making recycled content aromatics (r-aromatics) and recycled content paraxylene (r-paraxylene), and optionally, a recycled content organic chemical compound from the r-paraxylene, wherein the r-aromatics (and r-paraxylene and r-organic chemical compound) have physical content from one or more source materials;

FIG. 1b is a block flow diagram illustrating the main steps of a process for making recycled content aromatics (r-aromatics) and recycled content paraxylene (r-paraxylene), and optionally, a recycled content organic chemical compound from the r-paraxylene, wherein the r-aromatics (and r-paraxylene and r-organic chemical compound) have credit-based recycled content from one or more source materials;

FIG. 2 is a schematic block flow diagram illustrating the main processes/facilities in a system for providing recycled content organic chemical compounds, including r-paraxylene, r-terephthalic acid, and r-polyethylene terephthalate, according to various embodiments of the present invention;

FIG. 3 is a schematic block flow diagram illustrating the main steps/zones in a pyrolysis facility suitable for use in the system illustrated in FIG. 2;

FIG. 4 is a schematic block flow diagram illustrating the main steps/zones in a refinery suitable for use in the system illustrated in FIG. 2;

FIG. 5 is a schematic block flow diagram illustrating the main steps/zones in a refinery suitable for use in the system illustrated in FIG. 2, particularly illustrating possible locations of hydrotreating steps/units; and FIG. 6 is a schematic block flow diagram illustrating the main steps/zones in an aromatics complex suitable for use in the system illustrated in FIG. 2.

### DETAILED DESCRIPTION

We have discovered new methods and systems for producing paraxylene and organic chemical compounds formed by directly processing paraxylene or its derivatives, including, for example, organic chemical compounds such as terephthalic acid and polyethylene terephthalate. More specifically, we have discovered a process and system for producing paraxylene where recycled content from waste materials, such as waste plastic, are applied to paraxylene (or its derivatives) in a manner that promotes the recycling of waste plastic and provides paraxylene (or other organic chemical compounds) with substantial amounts of recycled content.

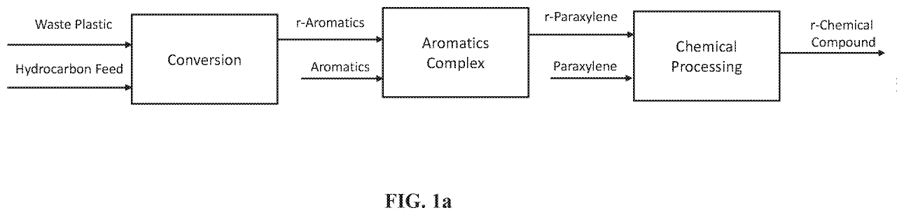

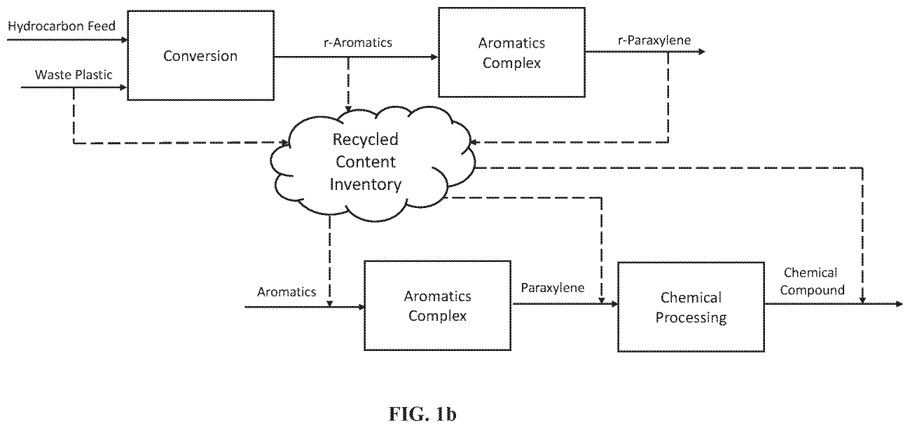

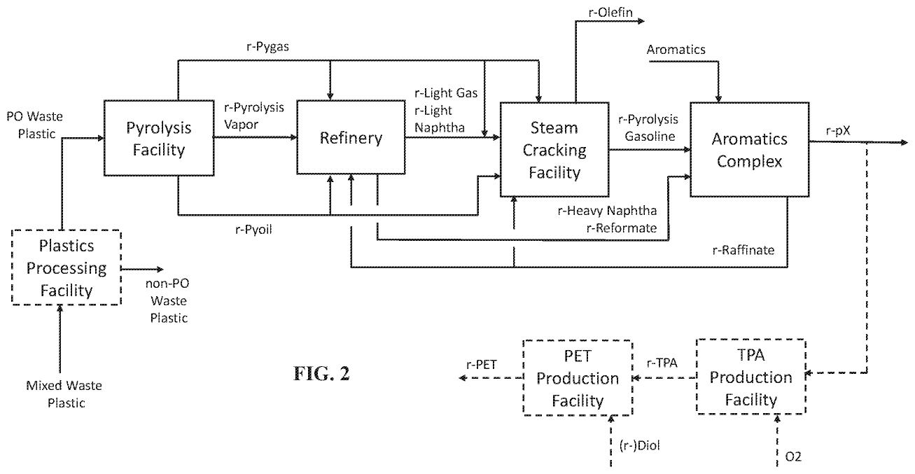

Turning initially to FIGS. 1a and 1b, paraxylene is formed by processing a predominantly aromatics stream in an aromatics complex to provide a stream including at least 85, at least 90, at least 92, at least 95, at least 97, or at least 99 weight percent paraxylene. The paraxylene stream can undergo one or more additional processing steps to provide at least one organic chemical compound derived from paraxylene. Examples of such organic chemical compounds include, but are not limited to, terephthalic acid, polymers such as polyethylene terephthalate, and other related organic chemical compounds.

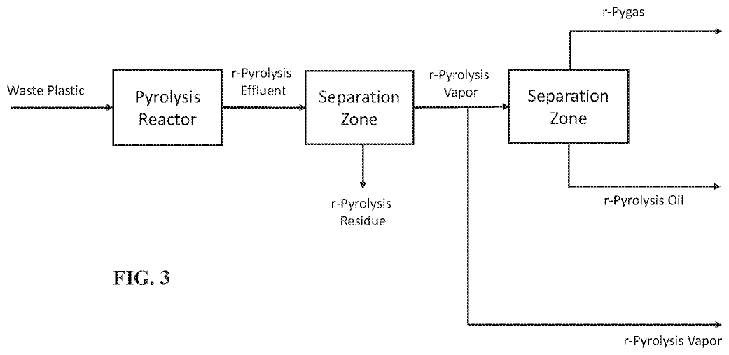

As generally shown in FIGS. 1a and 1b, a stream of waste plastic processed in one or more conversion facilities may provide the aromatics stream, which can be processed to form the paraxylene stream. The recycled content in the paraxylene stream can be physical and may directly originate from waste plastic or an intermediate hydrocarbon stream formed by processing waste plastic (not shown in FIG. 1 or 2), and/or the recycled content may be credit based and can be applied to a target stream in the aromatics complex and/or chemical processing facilities.

The aromatics (or paraxylene or organic chemical compound) streams can have a total recycled content of at least 5, at least 10, at least 15, at least 20, at least 25, at least 30, at least 35, at least 40, at least 45, at least 50, at least 55, or at least 65 percent and/or 100 percent, or less than 99, less than 95, less than 90, less than 85, less than 80, less than 75, or less than 70 percent. Similarly, the r-TPA and/or r-PET or even the r-aromatics stream can have a recycled content of at least 5, at least 10, at least 15, at least 20, at least 25, at least 30, at least 35, at least 40, at least 45, at least 50, at least 55, or at least 65 percent and/or 100 percent, or less than 99, less than 95, less than 90, less than 85, less than 80, less than 75, or less than 70 percent. The recycled content in one or more of these streams can be physical recycled content, credit-based recycled content, or a combination of physical and credit-based recycled content.

Turning initially to FIG. 1a, in one embodiment or in combination with one or more embodiments mentioned herein, at least a portion of the recycled content in the aromatics and/or paraxylene stream (or in the organic chemical compound product stream) can be physical (direct) recycled content. This recycled content may originate from a waste plastic stream. The waste plastic stream is ultimately converted in one or more conversion facilities (e.g., a pyrolysis facility, a refinery, a steam cracking facility, and/or a molecular reforming facility and methanol-to-aromatics facility), which is processed (alone or with a non-recycled content aromatics stream) as described herein to provide an r-paraxylene stream. The r-paraxylene stream can then be further processed (along or in combination with a non-recycled content paraxylene stream) to provide a recycled content organic chemical compound, including, but not limited to, recycled content terephthalic acid (r-TPA), recycled content polyethylene terephthalate (r-PET), and one or more additional recycled content organic chemical compounds (r-organic chemical compounds).

The amount of physical recycled content in the target product (e.g. composition, r-aromatics or r-paraxylene or r-organic chemical compound) can be determined by tracing the amount of waste plastic material processed along a chain of chemical pathway(s) and ending with the moiety or portion of the target product attributable to the waste plastic chemical pathway. As used herein, a moiety can be a portion the atoms and their structure of a target product and can also include the entire chemical structure of the target product, and does not necessarily require the inclusion of a functional group. For example, a moiety of p-xylene can include the aromatic ring, a portion of the aromatic ring, the methyl groups, or the entire p-xylene molecule. The chemical pathway includes all chemical reactions and other processing steps (e.g., separations) between the starting materials (e.g., waste plastic) and the moiety in the target product attributable to the chemical pathway originating in waste plastics. For example, the chemical pathway for the r-aromatics can include pyrolysis, optionally refining and/or stream cracking, and/or molecular reforming and methanol synthesis and conversion. The chemical pathway for the r-paraxylene can further include processing in the aromatics complex, and the chemical pathway for the r-organic chemical compound may include a variety of additional steps, such as, for example, oxidation, polymerization, etc., depending on the specific r-organic chemical compound. A conversion factor may be associated with each step along the chemical pathway. The conversion factors account for the amount of the recycled content diverted or lost at each step along the chemical pathway. For example, the conversion factors can account for the conversion, yield, and/or selectivity of the chemical reactions along the chemical pathway.

The amount of credit-based recycled content in a target product (e.g. compositions, r-aromatics or r-paraxylene or r-organic chemical compounds) can be determined by calculating the mass weight percent of a target moiety in a target product and attributing a recycle content credit to the target product in any amount up to the mass weight percent of the target moiety in the target product as a maximum. The credit based recycle content that is eligible to be applied to the target product is determined by tracing the waste plastic material along a chain of chemical pathway(s) and ending with the same moiety as target moiety in the target product. Thus, the credit based recycle content can be applied to a variety of different target products having the same moiety even though the products are made by entirely different chemical pathways provided that the credit applied is obtained from waste plastic and the waste plastic ultimately undergoes at least one chemical pathway originating from waste plastic and ending in the target moiety. For example, if a recycle content credit is obtained from waste plastic and booked into a recycle content inventory, and there exists chemical pathways at the facility capable of processing the waste plastic through to a target moiety such as p-xylene (e.g. a pyrolysis reactor effluent to a crude distillation column to a hydrotreater to a reformer to an aromatics complex that isolates p-xylene), the recycle content credit is then a type eligible to apply to any p-xylene molecule made by any chemical pathway, including the one existing at the facility and/or to the p-xylene portion of a pyrolysis gasoline stream composition obtained from a steam cracker and gasoline fractionator. As with physical recycled content, a conversion factor may or may not be associated with each step along the chemical pathway. Additional details on credit-based recycled content are provided below.

The amount of recycled content applied to the r-aromatics (or r-paraxylene or r-organic chemical compound) can be determined using one of variety of methods for quantifying, tracking, and allocating recycled content among various materials in various processes. One suitable method, known as “mass balance,” quantifies, tracks, and allocates recycled content based on the mass of the recycled content in the process. In certain embodiments, the method of quantifying, tracking, and allocating recycled content is overseen by a certification entity that confirms the accuracy of the method and provides certification for the application of recycled content to the r-aromatics (or r-paraxylene or r-organic chemical compound).

Turning now to FIG. 1b, one embodiment where the r-organic chemical compound (or r-paraxylene) includes credit-based recycled content, is provided. Recycled content credits from waste plastic are attributed to one or more streams within the facility. For example, the recycled content credits derived from waste plastics can be attributed to the aromatics stream fed to the aromatics complex, or to any of the products separated and isolated in the aromatics complex, such as to the paraxylene stream. Alternatively, or in addition, recycled content credits obtained from one or more intermediate streams within the conversion facility and/or aromatics complex can also be attributed to one or more products, such as paraxylene, within the facility, depending on the specific configuration of the system. Further, recycled content credits from one or more of these streams may also be attributed to the organic chemical compound stream, as shown in FIG. 1b.

As such, the waste plastic stream, or the r-aromatics stream and r-paraxylene streams (and any recycled content intermediate streams not shown in FIG. 1b) not made at the facility or purchased or acquired, can each act as a “source material” of recycled content credits. The aromatics fed to the aromatics complex, the paraxylene product or any other products separated and/or isolated from the aromatics complex, the paraxylene transferred (including sales) or fed to the chemical processing facility, any intermediate streams not shown, and even the organic chemical compound, can each act as a target product to which the recycled content credits are attributed. In one embodiment or in combination with any embodiment mentioned herein, the source material has physical recycled content and the target product has less than 100 percent physical recycled content. For example, the source material can have at least 10, at least 25, at least 50, at least 75, at least 90, at least 99, or 100 percent physical recycled content and/or the target product can have less than 100, less than 99, less than 90, less than 75, less than 50, less than 25, less than 10, less than 1 percent, or no physical recycled content.

The ability to attribute recycled content credits from a source material to a target product removes the co-location requirement between the facility making the source material (with physical recycled content) and the facility making the aromatics or products receiving recycle content value (e.g. paraxylene or organic chemical compound). This allows a chemical recycling facility/site in one location to process waste material into one or more recycled content source materials and then apply recycled content credits from those source materials to one or more target products being processed in existing commercial facilities located remotely from the chemical recycling facility/site, optionally within the same Family of Entities, or to associate a recycle content value with a product that is transferred to another facility, optionally owned by a different entity that can deposit the recycle content credit into its recycle content inventory one the product is receiving, purchased, or otherwise transferred. Further, the use of recycled content credits allows different entities to produce the source material and the aromatics (or paraxylene or organic chemical compound). This allows efficient use of existing commercial assets to produce the aromatics (or paraxylene or organic chemical compound). In one or more embodiments, the source material is made at a facility/site that is at least 0.1, at least 0.5, at least 1, at least 5, at least 10, at least 50, at least 100, at least 500, or at least 1000 miles from the facility/site where the target product is used to make the aromatics (or paraxylene or organic chemical compound).

The attributing of recycled content credits from the source material (e.g., the r-aromatics from the conversion facility) to the target product (e.g., an aromatics stream fed to an aromatics complex) can be accomplished by transferring recycled content credits directly from the source material to the target product. Alternatively, as shown in FIG. 1b, recycled content credits can be applied from any of the waste plastic, r-aromatics, and r-paraxylene (when present) to the aromatics, paraxylene, or organic chemical compound via a recycled content inventory.

When a recycled content inventory is used, recycled content credits from the source material having physical recycled content (e.g., the waste plastic, the r-aromatics, and optionally the r-paraxylene shown in FIG. 1b) are booked into the recycled content inventory. The recycled content inventory can also contain recycled content credits from other sources and from other time periods. In one embodiment, recycled content credits in the recycled content inventory correspond to a moiety, and the recycle content credit is applied or assigned to the same a target products containing a target moiety, and the target moiety is either (i) not chemically traceable through chemical pathways used to for generating the recycle content credit or (ii) is chemically traceable through chemical pathways used for generating the recycle content credit. Chemical traceability is achieved when atoms from a source material such as waste plastic can be theoretically traced to one or more atoms in the target moiety of a target product through each chemical pathway to obtain that atom(s) in the target moiety.

In some embodiments, there may be a periodic (e.g., annual or semi-annual) reconciliation between waste plastic credits deposited in the recycled content inventory and the mass of waste plastic processed. Such reconciliations may be performed by an appropriate entity at an interval consistent with rules of the certification system in which the producer is participating.

In one embodiment, once recycled content credits have been attributed to the target product (e.g., the aromatics stream, the paraxylene stream, or any intermediate stream not shown), the amount of the credit-based recycled content allocated to the organic chemical compound (e.g., TPA, PET, or other organic chemical compound) is calculated by the mass proportion of atoms in the target product that are chemically traceable to the source material. In another embodiment, a conversion factor can be associated with each step along the chemical pathway of the credit-based recycled content. The conversion factors account for the amount of the recycled content diverted or lost at each step along the chemical pathway. For example, the conversion factors can account for the conversion, yield, and/or selectivity of the chemical reactions along the chemical pathway. However, if desired, the amount of recycle content applied to a target product can be more than the mass proportion of the target moiety chemically traceable to the waste plastic source material. The target product can receive up to 100% recycle content even though the mass proportion of atoms in the target moiety that is chemically traceable to a recycle source material, such as mixed plastic waste stream, is less than 100%. For example, if the target moiety in a product represents only 30 wt. % of all atoms in a target product that are chemically traceable to a mixed plastic waste stream, the target product can nevertheless receive more than 30% recycle content value, up to 100% if desired. While such application would violate chemical traceability for the full value of the amount of recycle content in a target product back to a waste plastic source, the particular amount of recycle content value applied to a target product will depend on the rules of a certification system that the producer participates in.

As with the physical recycled content, the amount of credit-based recycled content applied to the r-aromatics (or r-paraxylene or r-organic chemical compound) can be determined using one of variety of methods, such as mass balance, for quantifying, tracing, and allocating recycled content among various products in various processes. In certain embodiments the method of quantifying, tracing, and allocating recycled content is overseen by a certification entity that confirms the accuracy of the method and provides certification for the application of recycled content to the r-aromatics (or r-paraxylene or r-organic chemical compound).

The r-aromatics (or r-paraxylene or r-organic chemical compound) can have 25 to 90, 40 to 80, or 55 to 65 percent credit-based recycled content and less than 50, less than 25, less than 10, less than 5, or less than 1 percent physical recycled content. In certain embodiments, the r-aromatics (or r-paraxylene or r-organic chemical compound) can have at least 10, at least 25, at least 50, or at least 65 percent and/or not more than 90, not more than 80, or not more than 75 percent credit-based recycled content from one or more of the r-aromatics and/or r-paraxylene, individually.

In one or more embodiments, the recycled content of the r-aromatics (or r-paraxylene or r-organic chemical compound) can include both physical recycled content and credit-based recycled content. For example, the r-aromatics (or r-paraxylene or r-organic chemical compound) can have at least 10, at least 20, at least 30, at least 40, or at least 50 percent physical recycled content and at least 10, at least 20, at least 30, at least 40, or at least 50 percent credit-based recycled content. As used herein, the term “total recycled content” refers to the cumulative amount of physical recycled content and credit-based recycled content from all sources.

Turning now to FIG. 2, a process and facility for use in forming a recycled content organic chemical compound is provided. As used herein, the term “organic chemical compound,” refers to a chemical compound that includes carbon and hydrogen atoms, but also includes oxygen and/or nitrogen atoms. An organic chemical compound can include at least 75, at least 80, at least 85, at least 90, at least 95, or at least 99 atom percent of carbon and hydrogen atoms combined, with the balance being nitrogen and oxygen.

Specifically, the system illustrated in FIG. 2 can form recycled content paraxylene (r-paraxylene) from one or more streams having recycled content derived from waste plastic. The system shown in FIG. 2 includes a pyrolysis facility, a refinery, a steam cracking facility, and an aromatics complex. Optionally, at least a portion of the r-paraxylene can be oxidized to form recycled content terephthalic acid (r-TPA) in a TPA production facility and at least a portion of the r-TPA can be reacted with at least one diol to form recycled content polyethylene terephthalate (r-PET) in a PET production facility. The r-paraxylene formed as described herein may be used in other applications not illustrated in FIG. 2.

Additionally, although not shown in FIG. 2, each of these facilities may also process a conventional stream of hydrocarbon-containing materials along with the waste plastic and/or stream derived from waste plastic. For example, the refinery may also process crude oil, the steam cracking facility may also process a hydrocarbon stream (e.g., light gas and/or naphtha), and the aromatics complex may also receive and process another aromatics-containing stream not from one or more of the conversion facilities. Further, the TPA and PET facilities may also process streams of paraxylene and/or terephthalic acid, respectively. These additional feed streams may or may not include recycled content.

The facility shown in FIG. 2 can be a chemical recycling facility. Chemical recycling facilities are not the same as mechanical recycling facilities. As used herein, the terms “mechanical recycling” and “physical recycling” refer to a recycling process that includes a step of melting waste plastic and forming the molten plastic into a new intermediate product (e.g., pellets or sheets) and/or a new end product (e.g., bottles). Generally, mechanical recycling does not substantially change the chemical structure of the plastic being recycled. The chemical recycling facilities described herein may be configured to receive and process waste streams from and/or that are not typically processable by a mechanical recycling facility.

In one embodiment or in combination with any embodiments mentioned herein, at least two, at least three, at least four, at least five, or all of the pyrolysis facility, the refinery, the steam cracking facility, the aromatics complex, and the optional TPA production facility and the optional PET production facility may be co-located. As used herein, the term “co-located” refers to the characteristic of at least two objects being situated on a common physical site, and/or within 5, within 3, within 1, within 0.75, within 0.5, or within 0.25 miles of each other, measured as a straight-line distance between two designated points.

When two or more facilities are co-located, the facilities may be integrated in one or more ways. Examples of integration include, but are not limited to, heat integration, utility integration, waste-water integration, mass flow integration via conduits, office space, cafeterias, integration of plant management, IT department, maintenance department, and sharing of common equipment and parts, such as seals, gaskets, and the like.

Additionally, one or more, two or more, three or more, four or more, five, or all, of the pyrolysis facility, the refinery, the steam cracking facility, the aromatics complex, the TPA production facility, and the PET production facility may be commercial-scale facilities. For example, in one embodiment or in combination with any embodiments mentioned herein, one or more of these facilities/steps can accept one or more feed streams at a combined average annual feed rate of at least 500, at least 1000, at least 1500, at least 2000, at least 5000, at least 10,000, at least 50,000, or at least 100,000 pounds per hour, averaged over one year. Further, one or more of the facilities can produce at least one recycled content product streams at an average annual rate of at least 500, or at least 1000, at least 1500, at least 2000, at least 2500, at least 5000, at least 10,000, at least 50,000, or at least 75,000 pounds per hour, averaged over one year. When more than one r-product stream is produced, these rates can apply to the combined rate of all r-products.

One or more, two or more, three or more, four or more, five, or all, of the pyrolysis facility, the refinery, the steam cracking facility, the aromatics complex, the TPA production facility, and the PET production facility can be operated in a continuous manner. For example, each of the steps or processes within each of the facilities and/or the process amongst the facilities may be operated continuously and may not include batch or semi-batch operation. In one embodiment or in combination with any embodiments mentioned herein, at least a portion of one or more of the facilities may be operated in a batch or semi-batch manner, but the operation amongst the facilities may be continuous overall.

As shown in FIG. 2, a stream of mixed waste plastic may be passed through an optional plastics processing facility. The plastics processing facility, when present, can separate mixed plastic into PET-enriched and a polyolefin (PO)-enriched streams and these separated streams can be introduced into separate conversion facilities. Additionally, or in the alternative, the plastics processing facility may also reduce the size of the incoming plastic via a crushing, flaking, pelletizing, grinding, granulating, and/or pulverizing step and/or the waste plastic may be melted or combined with a liquid to form liquified plastic or a slurry. One or more cleaning or separation steps may also be present to remove dirt, food, sand, glass, aluminum, lignocellulosic materials such as paper and cardboard, from the incoming waste stream.

As shown in FIG. 2, waste plastic, which can include PO-enriched waste plastic, can be introduced into the pyrolysis facility, wherein it may be pyrolyzed to form at least one recycled content pyrolysis effluent stream. In one embodiment or in combination with any embodiments mentioned herein, the system shown in FIG. 2 may also include a plastics processing facility for separating a stream of mixed plastic waste into a predominantly polyolefin (PO) waste plastic and a predominantly non-PO waste plastic, which typically includes waste plastics such as polyethylene terephthalate (PET), polyvinyl chloride (PVC), and others. In addition, when present, the plastics processing facility can also remove other non-plastic components, such as glass, metals, dirt, sand, and cardboard from the incoming waste stream.

Turning now to FIG. 3, a schematic diagram of the main steps/zones of a pyrolysis facility as shown in FIG. 2 are provided. As shown in FIG. 3, the waste plastic stream (including a predominantly PO waste plastic stream) can be introduced into a pyrolysis facility and pyrolyzed in at least one pyrolysis reactor. The pyrolysis reaction involves chemical and thermal decomposition of the waste plastic introduced into the reactor. Although all pyrolysis may be generally characterized by a reaction environment that is substantially free of molecular oxygen, pyrolysis processes may be further defined by other parameters such as the pyrolysis reaction temperature within the reactor, the residence time in the pyrolysis reactor, the reactor type, the pressure within the pyrolysis reactor, and the presence or absence of pyrolysis catalysts.

The feed to the pyrolysis reactor can comprise, consists essentially of, or consists of waste plastic, and the feed stream can have a number average molecular weight (Mn) of at least 3000, at least 4000, at least 5000, or at least 6000 g/mole. If the feed to the pyrolysis reactor contains a mixture of components, the Mn of the pyrolysis feed is the average Mn of all feed components, based on the weight of the individual feed components. The waste plastic in the feed to the pyrolysis reactor can include post-consumer waste plastic, post-industrial waste plastic, or combinations thereof. In certain embodiments, the feed to the pyrolysis reactor comprises less than 5, less than 2, less than 1, less than 0.5, or about 0.0 weight percent coal and/or biomass (e.g., lignocellulosic waste, switchgrass, fats and oils derived from animals, fats and oils derived from plants, etc.). The feed to the pyrolysis reaction can also comprise less than 5, less than 2, less than 1, or less than 0.5, or about 0.0 weight percent of a co-feed stream, including steam and/or sulfur-containing co-feed streams. In other cases, steam fed to the pyrolysis reactor can be present in amounts of up to 50 weight percent.

The pyrolysis reaction can involve heating and converting the waste plastic feedstock in an atmosphere that is substantially free of molecular oxygen or in an atmosphere that contains less molecular oxygen relative to ambient air. For example, the atmosphere within the pyrolysis reactor may comprise not more than 5, not more than 4, not more than 3, not more than 2, not more than 1, or not more than 0.5 weight percent of molecular oxygen.

The pyrolysis reaction in the reactor can be thermal pyrolysis, which is carried out in the absence of a catalyst, or catalytic pyrolysis, which is carried out in the presence of a catalyst. When a catalyst is used, the catalyst can be homogenous or heterogeneous and may include, for example, oxides, certain types of zeolites, and other mesostructured catalysts.

The pyrolysis reactor may have any suitable design and can comprise a film reactor, a screw extruder, a tubular reactor, a stirred tank reactor, a riser reactor, a fixed bed reactor, a fluidized bed reactor, a rotary kiln, a vacuum reactor, a microwave reactor, or an autoclave. The reactor may also utilize a feed gas and/or lift gas for facilitating the introduction of the feed into the pyrolysis reactor. The feed gas and/or lift gas can comprise nitrogen and can comprise less than 5, less than 2, less than 1, or less than 0.5, or about 0.0 weight percent of steam and/or sulfur-containing compounds. The feed and/or lift can also include light hydrocarbons, such a methane, or hydrogen, and these gases may be used alone or in combination with steam.

As shown in FIG. 3, a stream of recycled content pyrolysis effluent (r-pyrolysis effluent) removed from the reactor can be separated in a separation zone to provide a recycled content pyrolysis vapor (r-pyrolysis vapor) stream and a recycled content pyrolysis residue (r-pyrolysis residue) stream. The r-pyrolysis vapor can include a range of hydrocarbon materials and may comprise both recycled content pyrolysis gas (r-pygas) and recycled content pyrolysis oil (r-pyoil). In some embodiments, the pyrolysis facility may include an additional separation zone, as shown in FIG. 3, to separate the r-pyoil and r-pygas into separate streams. Alternatively, the entire stream of r-pyrolysis vapor may be withdrawn from the pyrolysis facility and routed to one or more downstream processing facilities.

The r-pyoil can include predominantly C5 to C22 hydrocarbon components, or it can include at least 55, at least 60, at least 65, at least 70, at least 75, or at least 80 weight percent of C 5 to C 22 hydrocarbon components, while the r-pygas can include predominantly C2 to C 4 hydrocarbon components, or at least 30, at least 40, at least 45, at least 55, at least 60, at least 65, at least 70, at least 75, or at least 80 weight percent C2 to C 4 hydrocarbon components. In some cases, the C2 to C 4 components in the r-pygas can include at least 50, at least 55, at least 60, at least 65, at least 70, or at least 75 weight percent of alkanes and/or at least 40, at least 45, at least 50, at least 55, at least 60, at least 65, at least 70, or at least 75 weight percent of olefins, based on the amount of C2 to C4 hydrocarbon components in the stream.

The r-pyoil may also comprise one or more of the following (i) through (v): (i) less than 500 ppm, less than 450 ppm, less than 350 ppm, less than 250 ppm, less than 100 ppm, less than 75 ppm, less than 50 ppm, less than 25 ppm, or less than 10 ppm sulfur; (ii) less than 300 ppm, less than 150 ppm, less than 100 ppm, less than 50 ppm, less than 25 ppm, less than 10 ppm, or less than 5 ppm chlorine; (iii) less than 500 ppm, less than 250 ppm, less than 100 ppm, less than 75 ppm, less than 50 ppm, less than 30 ppm, or less than 20 ppm water; (iv) less than 500 ppb, less than 250 ppb, less than 100 ppb, less than 50 ppb, less than 25 ppb, less than 10 ppb, less than 5 ppb, or less than 2 ppb arsenic; and/or (v) less than 1500 ppm, less than 1000 ppm, less than 500ppm, less than 250 ppm, less than 100 ppm, less than 75 ppm, less than 50 ppm, less than 30 ppm, or less than 20 ppm nitrogen.

As also illustrated in FIG. 2, at least a portion of the r-pyoil, r-pygas, and/or r-pyrolysis vapor can be introduced into a refinery, wherein one or more of these streams can undergo one or more processing steps to provide at least a stream of recycled content light gas (r-light gas) and/or a stream of recycled content naphtha (r-naphtha), as well as one or more other recycled content hydrocarbon streams. Examples of suitable processing steps include, but are not limited to, distillation or other separation steps as well as chemical processing such as thermal and/or catalytic cracking or other reactions such as reforming and isomerization.

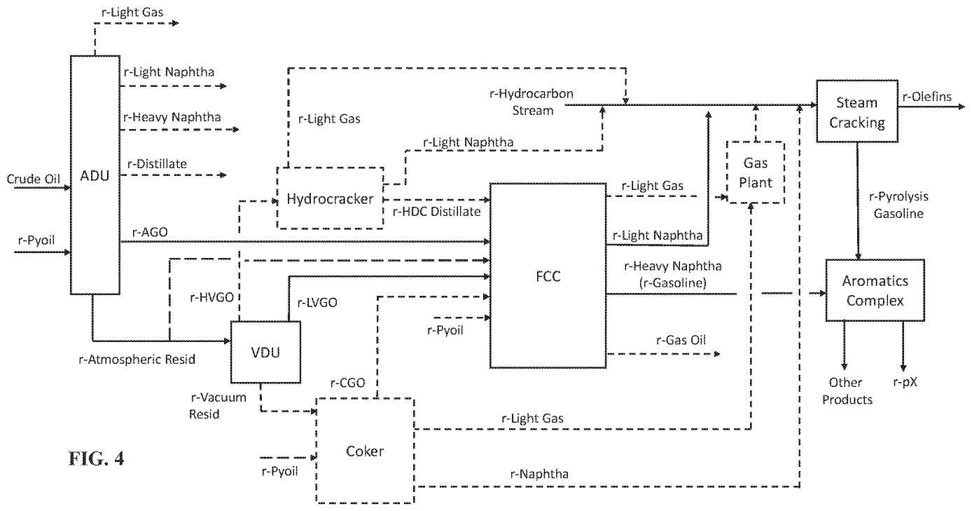

Turning now to FIG. 4, a schematic diagram of the main steps or zones in a refining facility, or refinery, suitable for processing at least one hydrocarbon stream including recycled content derived from waste plastic is provided. It should be understood that other processing steps may exist and/or other recycled content hydrocarbon streams may be produced in the refinery shown in FIG. 4. The steps, zones, and process streams illustrated in FIG. 4 are provided for simplicity and not intended to exclude other steps, zones, or process streams not shown.

As shown in FIG. 4, a stream of crude oil may be introduced into an atmospheric distillation unit (ADU) and separated in at least one distillation column to provide several hydrocarbon fractions having specified cut points. As used herein, the term “cut point” refers to the range of temperatures over which a specified petroleum fraction boils. The lower value in a boiling point range is the initial boiling point (IBP) temperature for that specified fraction and the higher value is the end point (EP) temperature for that specified fraction. Cut points are often used to identify specific streams or fractions within and/or produced by the refinery.

In addition to a stream of crude oil, the refinery shown in FIG. 4 can also process a stream of r-pyoil introduced into the ADU. In one embodiment or in combination with any embodiments mentioned herein, the r-pyoil may originate from a pyrolysis as discussed previously with respect to FIG. 2. The r-pyoil introduced into the ADU can comprise less than 50, less than 40, less than 30, less than 20, less than 10, less than 9, less than 8, less than 7, less than 6, less than 5, less than 4, less than 3, less than 2, or less than 1 weight percent of the total feed to the at least one distillation column.

The ratio of the mass flow rate of r-pyoil introduced into the ADU to the mass flow rate of petroleum oil introduced into the ADU can be at least 1:1000, at least 1:750, at least 1:500, at least 1:250, at least 1:100, at least 1:50, at least 1:25, or at least 1:10 and/or not more than 1:1, not more than 1:2, not more than 1:5, or not more than 1:10. The amount of r-pyoil introduced into the ADU can be at least 0.1, at least 0.25, at least 0.75, at least 1, at least 5, at least 10, at least 15, at least 20 weight percent and/or not more than 75, not more than 65, not more than 60, not more than 50, or not more than 45 weight percent of the total feed to the at least one distillation column.

Alternatively, when the r-pyoil is not introduced into the ADU, the feed to the atmospheric distillation column may include less than 1000, less than 500, less than 250, less than 100, less than 75, less than 50, less than 30, or less than 20 parts per million (ppm) by weight of r-pyoil, or it can include no r-pyoil. Additionally, or in the alternative, a stream of recycled content pyrolysis vapor (r-pyrolysis vapor) and/or a stream of recycled content pyrolysis residue (r-pyrolysis residue) could be introduced into the ADU, alone or in combination with one another and/or r-pyoil and may be further separated as described herein.

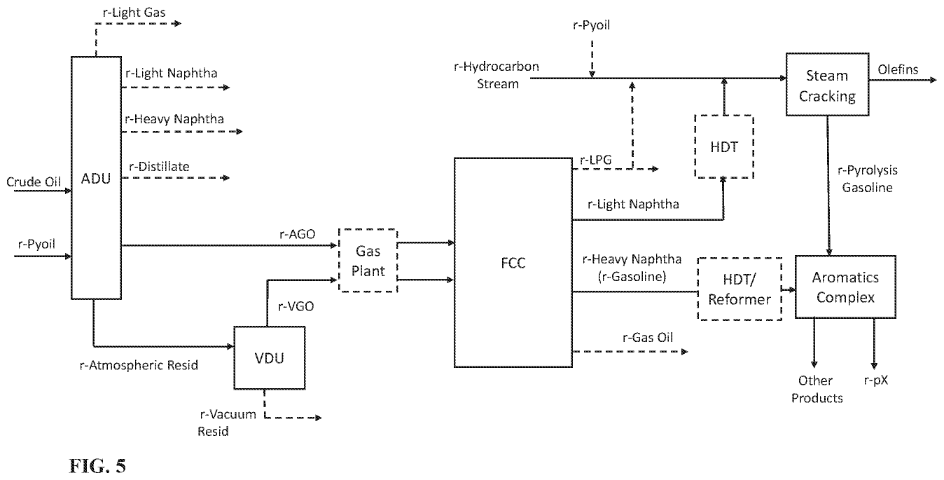

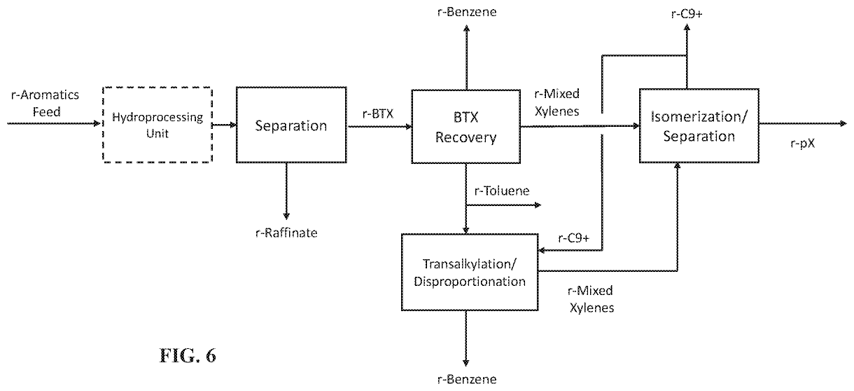

The crude oil and/or r-pyoil (or other r-pyrolysis stream introduced into the ADU) can include one or more compounds in addition to hydrocarbons including, for example, sulfur-containing compounds, chlorine-containing compounds, nitrogen-containing compounds, and metals. One or more processing steps within the refinery can be used to remove one or more of these components, as discussed in detail below with respect to FIG. 5. In some cases, the chlorine content of the r-pyoil (or other r-pyrolysis stream) may be higher than conventional hydrocarbon feed to the ADU. For example, the total content of chlorine-containing compounds in the r-pyoil (or combined r-pyoil and crude oil) stream can be at least 20, at least 50, at least 75, at least 100 ppm by weight and/or not more than 500, not more than 350, not more than 200, or not more than 100 ppm by weight.

The ADU separates feed stock (e.g., crude oil) into multiple hydrocarbon streams, or fractions. As shown in FIG. 4, these fractions include, but are not limited to, light gas, naphtha, distillate, gas oil (called atmospheric gas oil, or AGO), and residue or resid. When the ADU processes at least one recycled content feedstock, such as r-pyoil and/or r-pyrolysis vapor, each of the products formed by the ADU may include recycled content. Thus, as shown in FIG. 4, the ADU may provide recycled content light gas (r-light gas), recycled content light naphtha (r-naphtha), recycled content heavy naphtha (r-heavy naphtha), recycled content distillate (r-distillate), recycled content atmospheric gas oil (r-AGO), and recycled content atmospheric resid (r-atmospheric resid). The mass flow rate of each stream, as well as its mass or volume in proportion to other streams, depends on the operation of the ADU as well as the properties of the feedstocks being processed. As mentioned previously, other hydrocarbon streams can be produced from the ADU, but are not shown here for simplicity.

The ADU comprises at least one distillation column operated at or near atmospheric pressure. Additionally, the ADU may include other equipment such as desalters, side strippers, and reflux drums/accumulators, as well as various pumps, heat exchangers, and other auxiliary equipment needed to operate the unit.

As shown in FIG. 4, the overhead gas stream (e.g., r-light gas) withdrawn from the ADU includes comprises predominantly C6 and lighter components. In one embodiment or in combination with any embodiments mentioned herein, this predominantly gas stream can include at least 55, at least 60, at least 65, at least 70, at least 75, at least 80, at least 85, at least 90, or at least 95 weight percent of C6 and lighter components. In one embodiment or in combination with any embodiments mentioned herein, this stream may also include at least 25, at least 30, or at least 35 weight percent of C1 and lighter components, as well as small amounts of sulfur-containing compounds, chlorine-containing compounds, and/or nitrogen-containing compounds. As used herein, the term “C1 and lighter” refers to methane (C1) and compounds having a lower boiling point than methane, at standard conditions. Examples of components lighter than C1 include, but are not limited to hydrogen (H2), carbon monoxide (CO), and nitrogen (N2).

The overhead r-light gas stream from the ADU may be processed in a saturated gas plant (not shown), wherein it can be separated into two or more streams via one or more distillation steps. For example, in one embodiment or in combination with any embodiments mentioned herein, the overhead gas stream may be separated to remove most of the C1 and lighter components in a demethanizer column, and/or may be processed to remove most of the C5 and heavier components in a debutanizer column. Other columns (e.g., deethanizer, depropanizer, etc.) may also be used to form various product streams (e.g., ethane, propane, etc.) depending on the configuration of the refinery and the saturated gas plant. The saturated gas plant can also include one or more treatment steps for the removal of nitrogen-, chlorine-, and/or sulfur-containing components.

As also shown in FIG. 4, streams of r-light naphtha and r-heavy naphtha and r-distillate may be withdrawn from the ADU and can be sent to one or more downstream locations for additional processing, storage, and/or use. For example, at least a portion of the r-light naphtha and/or r-heavy naphtha may be sent to a catalytic reformer to provide a stream of recycled content reformate (r-reformate), which may comprise predominantly C6 to C10 aromatics. All or a portion of this can be introduced into an aromatics complex, as discussed in further detail shortly. One or more of these streams may also be further processed to remove components such as sulfur-containing compounds, chlorine-containing compounds, and/or nitrogen before further processing and/or use. Prior to introduction into the reformer, all or a portion of the r-light and/or r-heavy naphtha may be hydrotreated, or it may not be hydrotreated.

The heaviest stream withdrawn from the ADU is a stream of recycled content atmospheric resid (r-atmospheric resid). In some cases, the r-atmospheric resid may be sent directly to a fluidized catalytic cracker (FCC), while in other cases, it may be introduced into a vacuum distillation unit (VDU). In the VDU, further separation of various hydrocarbon fractions can be performed in a vacuum distillation column operated at pressures below atmospheric pressure. For example, in one embodiment or in combination with any embodiments mentioned herein, the overhead pressure of the vacuum distillation column can be less than 100, less than 75, less than 50, less than 40, or less than 10 mm Hg. Distilling the r-atmospheric resid at low pressure permits further recovery of lighter hydrocarbon components without cracking. The VDU provides product streams similar to the ADU, and when it processes a recycled content feedstock, provides recycled content products. Examples of such products include, but are not limited to, recycled content light vacuum gas oil (r-LVGO), recycled content heavy vacuum gas oil (r-HVGO), and recycled content vacuum resid (r-vacuum resid).

In one embodiment or in combination with any embodiments mentioned herein, at least a portion of one or more of the heavier hydrocarbon fractions (e.g., heavier than distillate) from the ADU and/or VDU can be sent to a gas oil cracker. Such heavier hydrocarbon fractions can have a median boiling point (T50) greater than 375, greater than 400, greater than 500, greater than 600, greater than 650, greater than 700, greater than 800, or greater than 900 °F and/or not more than 1050, not more than 1000, not more than 950, not more than 900, not more than 850, not more than 800, not more than 700, not more than 650 °F, or in the range of from 400 °F to 1050 °F, from 500 °F to 1000 °F, or 650 °F to 800 °F, or it can be in the range of from 375 to 800 °F, or 400 °F to 650 °F. One or more of these heavy hydrocarbon fractions can comprise at least 85, at least 90, at least 95, at least 97, or at least 99 weight percent of C 10, C15, C20, or C25 and heavier components. Examples of these streams as shown in FIG. 4 can include, but are not limited to, r-AGO, r-atmospheric resid, r-vacuum resid, r-LVGO, and r-HVGO.

As mentioned previously, at least a portion of the r-atmospheric resid may be introduced into the FCC, particularly when the refinery does not include a VDU. As shown in FIG. 4, when the refinery has a VDU, at least a portion of the r-vacuum resid may be fed to the coker, while the r-HVGO may be introduced into the hydrocracker (HDC). The r-LVGO can be fed to the FCC along with at least a portion of the r-AGO. Depending on the specific configuration of the refinery, other processing schemes are possible that fall within the scope of the present technology. The r-LVGO may or may not be process in a hydrotreater prior to entering the FCC or other gas oil cracker.

The gas oil cracker can be any processing unit or zone that reduces the average molecular weight of a heavy hydrocarbon feedstock to provide one or more lighter hydrocarbon products (e.g., naphtha, light gas, etc.) via thermal and/or catalytic cracking. Gas oil crackers may be operated at temperatures of at least 350 °F, at least 400 °F, at least 450 °F, at least 500 °F, at least 550 °F, or at least 600 °F and/or not more than 1200 °F, not more than 1150 °C, not more than 1100 °F, not more than 1050 °F, not more than 1000 °F, not more than 900 °F, or not more than 800 °F. Gas oil crackers may be operated at or near atmospheric pressure (e.g., at a pressure of less than 5 psig, less than 2 psig, or 1 psig) or may be operated at elevated pressure (e.g., at a pressure of at least 5 psig, at least 10 psig, at least 25 psig, at least 50 psig, at least 100 psig, at least 250 psig, at least 500 psig, or at least 750 psig.) Additionally, the cracking in gas oil crackers may be carried with or without a catalyst, and it may or may not be conducted in the presence of hydrogen and/or steam. The gas oil cracker may include other equipment such as compressors, distillation columns, heat exchangers, and other equipment necessary to provide the cracked product streams. Examples of gas oil crackers illustrated in FIG. 4 include a fluidized catalytic cracker (FCC), a coker, and a hydrocracker (HDC).

Alternatively, or in addition, at least a portion of the cracking can be performed in the presence of hydrogen (e.g., in a hydrocracker as shown in FIG. 4) so that removal of components such as nitrogen-, chlorine-, and sulfur-containing components (and optionally metals), may be performed at the same time as the cracking reaction. When cracking and hydrogenation occur simultaneously, the saturation of olefinic hydrocarbons may also take place such that the amount of or olefins in the hydrocracker product stream is not more than 20, not more than 15, not more than 10, or not more than 5 weight percent. However, aromatics may remain such that the amount of aromatics in the stream withdrawn from the hydrocracker may be at least 1, at least 5, at least 10, at least 20, or at least 25 and/or not more than 50, not more than 40, or not more than 35 weight percent.

In one embodiment or in combination with any embodiments mentioned herein, one or more recycled content cracked hydrocarbon (r-cracked hydrocarbon) streams from the gas oil cracker may be further cracked in another gas oil cracker to provide additional recycled content cracked hydrocarbon (r-cracked hydrocarbon) streams. For example, as shown in FIG. 4, at least a portion of the r-LVGO from the VDU and/or the r-hydrocracker distillate (r-HDC) from the hydrocracker may be introduced into the FCC. Additionally, or in the alternative, at least a portion of a stream of recycled content coker gas oil (r-CGO) from the coker can be introduced into the FCC for further cracking to form r-light gas and/or r-naphtha. Other processing schemes are possible depending on the specific equipment and configuration of the refinery.

In one embodiment or in combination with any embodiments mentioned herein, a stream of r-pyoil and/or waste plastic (not shown) can be directly introduced into one or more gas oil cracker units within the refinery. For example, a stream of r-pyoil and/or waste plastic may be directly fed into at least one (at least two, or each) of a coker, a hydrocracker, and an FCC. These streams may be co-fed with one or more other hydrocarbon streams, which may or may not include recycled content. For example, as shown in FIG. 4, r-pyoil fed to the FCC may be co-fed with r-AGO, r-LVGO, r-CGO, r-atmospheric resid, and/or r-HDC distillate. One or more of the gas oil and/or distillate streams fed to the gas oil cracker may also include non-recycled content, particularly if no r-pyoil (or other r-pyrolysis stream) is fed to the ADU.

When waste plastic is fed to one of these gas oil crackers, the waste plastic may be liquified mixed plastic waste formed by heating the waste plastic to at least partially melt it and/or by combining waste plastic with at least one solvent such as gas oil, r-gas oil, and/or r-pyoil. When combined with a solvent, the waste plastic may be dissolved or it may be in the form of a slurry. In one embodiment or in combination with any embodiments mentioned herein, the waste plastic introduced into the refinery may not have been separated (e.g., it may be mixed plastic waste), while in other cases, it may have undergone at least one separation step so that the waste plastic comprises predominantly polyolefin (PO) waste plastic. In such cases, the waste plastic may include at least 55, at least 60, at least 65, at least 70, at least 75, at least 80, at least 85, at least 90, at least 95, or at least 99 weight percent of PO waste plastic, based on the total weight of the stream.

In one embodiment or in combination with any embodiments mentioned herein, at least a portion of a recycled content effluent (r-effluent) stream from the FCC, hydrocracker, and/or coker reaction vessels may be separated into several recycled content cracked hydrocarbon fractions, including a recycled content light gas (r-light gas) stream, a recycled content light naphtha stream (r-light naphtha), a recycled content heavy naphtha (r-heavy naphtha) stream, recycled content cracked distillate (r-cracked distillate) stream, and a recycled content gas oil (r-gas oil) stream.

For example, as shown in FIG. 4, at least a portion of the r-cracked effluent from the FCC can be separated in a gas plant, alone or in combination with a stream of r-light gas from the coker. (In some cases, the FCC and coker may have separate gas plants.) At least a portion of this separation may be performed in an unsaturated gas plant, such as a coker gas plant or an FCC gas plant, or it may be carried out in a saturated gas plant, such as a column in an HDC unit or in the saturated gas plant for processing the atmospheric distillation column overhead stream. When the r-effluent includes at least 15, at least 20, at least 25, or at least 30 weight percent of olefins, at least a portion may be separated in one or more unsaturated gas plants and when the r-effluent includes less than 15, less than 10, less than 5, or less than 2 weight percent of olefinic compounds, it may be processed in at least one saturated gas plant.

In one embodiment or in combination with any embodiments mentioned herein, one or more of the r-light gas streams shown in FIG. 4 can comprise at least 55, at least 60, at least 65, at least 70, at least 75, at least 80, at least 85, at least 90, or at least 95 weight percent C3 and lighter or C2 and lighter components. The r-light gas streams may include at least 15, at least 20, at least 25, or at least 30 and/or not more than 50, not more than 45, not more than 40, or not more than 35 weight percent of C1 and lighter components and/or less than 20, less than 15, less than 10, less than 5, less than 2, less than 1, less than 0.5 or less than 0.1 weight percent of C4 and heavier components.

In one embodiment or in combination with any embodiment mentioned herein, at least a portion of the r-cracked effluent from a gas oil cracker can be separated into streams of r-light naphtha and r-heavy naphtha. As used herein, the term “light naphtha” refers to a specific portion of a naphtha cut in a refinery having a boiling point range between 90 and less than 230 °F. As used herein, the term “heavy naphtha” refers to a specific portion of a naphtha cut in a refinery having a boiling point range between 230 and 380 °F.

The r-light naphtha predominantly comprises C5 and C6 hydrocarbons and has a boiling point range of at least 90, at least 95, or at least 100 °F and/or less than 230, not more than 225, or not more than 220 °F and/or a T50 boiling point of at least 95, at least 100, or at least 110 °F and/or not more than 185, not more than 180, or not more than 175 °F. The r-light naphtha can include olefins in an amount of from 0.001 to 25 weight percent, from 0.01 to 10 weight percent, or from 0.1 to 5 weight percent, and it can include alkanes (including cycloalkanes) in an amount of from 70 to 99 weight percent, from 80 to 95 weight percent, or at least 70, at least 80, at least 90, or at least 95 weight percent. The r-light naphtha may also comprise aromatic hydrocarbons in an amount of from 0.1 to 15 weight percent, from 0.2 to 10 weight percent, from 0.5 to 5 weight percent, or less than 10 weight percent, less than 5 weight percent, less than 2 weight percent, or less than 1 weight percent aromatic hydrocarbon compounds. Additionally, the r-light naphtha can include from 0.1 to 20 weight percent, 0.1 to 10 weight percent, or 0.5 to 5 weight percent cycloparaffins and/or naphthenes, or less than 20 weight percent, less than 10 weight percent, less than 5 weight percent, less than 2 weight percent, or less than 1 weight percent of cycloalkanes (e.g., cycloparaffins and/or naphthenes).

The r-heavy naphtha predominantly comprises C6 and heavier, or C7 to C15 hydrocarbons and has a boiling point range of at least 230, at least 235, or at least 240 °F and/or less than 380, not more than 375, or not more than 370 °F. The r-heavy naphtha can include at least 55, at least 65, at least 75, at least 85, or at least 90 weight percent of C6 and heavier or C7 and heavier components, and may include at least 20, at least 25, at least 30, at least 35, at least 40, or at least 45 weight percent and/or not more than 95, not more than 90, not more than 80, not more than 75, not more than 70, not more than 65, not more than 60, or not more than 55 weight percent of C6 to C10 components. At least a portion of the C6 to C10 components can include aromatics, such that, for example, the r-heavy naphtha stream includes an amount of C6 to C10, or C6 to C9, or C6 to C8 aromatics within one or more of the above ranges.

In one embodiment or in combination with any embodiments mentioned herein, at least one of the heavy hydrocarbon streams introduced into and/or at least one of the cracked hydrocarbon streams removed from one or more of the gas oil crackers (e.g., hydrocracker, coker, and/or FCC) may be treated with hydrogen to remove all or a portion of one or more components such as sulfur-containing compounds (e.g., thioethers, thiophene, mercaptans, etc.), nitrogen-containing compounds (e.g., pyrroles, pyridines, etc.), metals (e.g., vanadium, mercury, etc.), and/or chlorine-containing compounds (e.g., chloroalkanes, etc.) and/or to saturate at least a portion of the olefinic and/or aromatic compounds in the stream.

For example, turning now to FIG. 5, at least a portion of the r-AGO and/or r-atmospheric resid from the ADU and/or the r-VGO and/or r-vacuum resid from the VDU (or any other heavy oil steam not shown in FIG. 5) may be processed in at least one hydrotreating (HDT) step/unit prior to being introduced into the FCC. The hydrotreating step/unit may utilize any suitable process for removing all or a portion of one or more components described above and/or to saturate at least a portion of the olefinic and/or aromatic compounds in the stream. The hydrotreating process may be performed at temperatures in the range of 250 to 500 °C, or 300 to 450 °C and pressures of about 250 to over 2000 psig, or 300 to 1500 psig, depending on the feed and desired product composition. Typically, the hydrotreating process utilizes at least one catalyst. Examples of suitable catalysts include, but are not limited to, one or more metals such as copper, zinc, cobalt, molybdenum, nickel and/or tungsten on a support comprising one or more of alumina, silica, aluminosilicate, and molecular sieve. Alternatively, the r-AGO and/or r-VGO (or other heavy oil stream not shown) may not be hydrotreated prior to being introduced into the FCC.

Additionally, or in the alternative, at least a portion of the r-light naphtha can be hydrotreated prior to introduction into the steam cracking facility and/or at least a portion of the r-heavy naphtha can be hydrotreated prior to introduction into the aromatics complex. Additionally, as shown in FIG. 5, a stream of r-pyoil from a pyrolysis facility may also be introduced into the steam cracking facility. In some cases, the r-pyoil introduced into the steam cracking facility can comprise light r-pyoil and may have been separated prior to entering the inlet of the steam cracking furnace. Light r-pyoil may comprise predominantly C5 to C8, C5 to C7 or C5 and C6 compounds and may be separated in or just downstream of the pyrolysis facility (not shown in FIG. 5). Alternatively, one or both of the r-light naphtha and r-heavy naphtha streams may not be hydrotreated prior to entering the steam cracking facility and/or the aromatics complex.

As shown in both FIGS. 1 and 4, at least a portion of one or more of the r-light gas and/or r-naphtha streams from the refinery and/or at least a portion of the r-pygas and/or r-pyoil from the pyrolysis facility can be introduced into a steam cracking facility to provide a recycled content pyrolysis gasoline (r-pyrolysis gasoline) stream. For example, as shown in FIG. 4, at least a portion of the r-light gas stream from the hydrocracker and/or from a gas plant associated with the FCC and/or coker. Additionally, or in the alternative, at least a portion of the r-light naphtha from the hydrocracker and/or at least a portion of the r-light naphtha from the FCC may also be introduced into the steam cracking facility.

In some cases, gas-phase streams (e.g., r-pygas and/or r-light gas, optionally with another predominantly C2 to C4 gas stream with or without recycled content) can be introduced into the inlet of a steam cracker furnace in the steam cracking facility, while, in other cases, these streams may be introduced into one or more locations downstream of the furnace. When one or more liquid-phase streams (e.g., r-pyoil and/or r-light naphtha, optionally with another liquid hydrocarbon stream of similar composition with or without recycled content) are introduced into the steam cracking facility, these streams can be fed to the inlet of the steam cracking furnace.

In the steam cracking furnace, the hydrocarbon feed stream, which can include one or more of r-pygas, r-pyoil, r-light gas, and r-light naphtha as well as other recycled and/or non-recycled content hydrocarbon, can be thermally cracked in the presence of steam to form a predominantly recycled content olefin (r-olefin-containing) stream and a stream of recycled content pyrolysis gasoline (r-pyrolysis gasoline). The r-olefin-containing stream may be compressed and further processed in a separation zone of the steam cracking facility to provide one or more recycled content olefins (r-olefins), such as, r-ethylene and/or r-propylene, while a recycled content pyrolysis gasoline (r-pyrolysis gasoline), which comprises predominantly C6 to C10 aromatics, can be withdrawn from the steam cracking facility and introduced into the aromatics complex as shown in FIG. 2. All or a portion of the r-pyrolysis gasoline may be hydrotreated prior to entering the aromatics complex, or all or a portion may not.

The r-pyrolysis gasoline stream comprises at least 20, at least 25, at least 30, at least 35, at least 40, at least 45, or at least 50 weight percent and/or not more than 85, not more than 80, not more than 75, not more than 70, not more than 65, or not more than 60 weight percent of recycled content benzene, recycled content toluene, and recycled content xylenes (r-BTX). In one embodiment or in combination with any embodiments mentioned herein, the r-pyrolysis gasoline can also include at least 5, at least 10, or at least 15 weight percent and/or not more than 45, not more than 35, not more than 30, or not more than 25 weight percent of recycled content C9 to C12 aromatics and/or recycled content C6 and heavier cyclic hydrocarbons (r-C6+cyclic hydrocarbons).

The r-pyrolysis gasoline can include at least 1, at least 5, at least 10, at least 15 and/or not more than 30, not more than 25, not more than 20, not more than 15, or not more than 10 weight percent of styrene. Or, at least a portion of the styrene may be removed from the r-pyrolysis gasoline so that it includes not more than 5, not more than 2, not more than 1, or not more than 0.5 weight percent styrene. Additionally, or in the alternative, the r-pyrolysis gasoline can include at least 0.01, at least 0.05, at least 0.1, or at least 0.5 and/or not more than 5, not more than 2, not more than 1, or not more than 0.75 weight percent of one or more of cyclopentadiene and dicyclopentadiene.

In one embodiment or in combination with any embodiments mentioned herein, the r-pyrolysis gasoline can include at least 5, at least 10, at least 15, at least 20, at least 25, at least 30, at least 35, at least 40, or at least 45 weight percent and/or not more than 55, not more than 50, not more than 45, or not more than 40 weight percent of benzene, and/or at least 5, at least 10, at least 15, at least 20, at least 25, or at least 30 weight percent and/or not more than 35, not more than 30, not more than 25, not more than 20, not more than 15, or not more than 10 weight percent of toluene, based on the total amount of BTX or the total amount of the r-pyrolysis gasoline stream. Additionally, or in the alternative, the r-pyrolysis gasoline can include at least 1, at least 2, at least 5, or at least 7 weight percent and/or not more than 20, not more than 15, or not more than 10 weight percent of mixed xylenes, including ortho-xylene (oX), meta-xylene (mX), and paraxylene (pX), based on the total amount of BTX or the total amount of the r-pyrolysis gasoline stream. At least a portion of the benzene, toluene, and/or xylenes in the r-pyrolysis gasoline can comprise recycled content benzene, recycled content toluene, and/or recycled content xylenes, while, in other cases, at least a portion of the benzene, toluene, and/or xylenes may include non-recycled content.

Additionally, or in the alternative, the pyrolysis gasoline (or r-pyrolysis gasoline) can include at least 1, at least 2, at least 5, or at least 10 weight percent and/or not more than 25, not more than 20, not more than 15, or not more than 10 weight percent of other C8 aromatics, such as ethylbenzene. The pyrolysis gasoline can also include at least 1, at least 2, at least 5, or at least 10 weight percent and/or not more than 25, not more than 20, not more than 15, not more than 10, or not more than 7 weight percent of C9 and/or C10 aromatics, based on the total weight of the stream. The pyrolysis gasoline may also include little or no C5 and lighter and/or C11 and heavier components, such that these may be present in an amount of not more than 10, not more than 5, not more than 2, or not more than 1 weight percent.

Turning again to FIG. 2, at least a portion of the r-pyrolysis gasoline withdrawn from the steam cracking facility and/or at least one r-reformate stream and/or r-heavy naphtha from the refinery can be introduced into an aromatics complex. These streams may be introduced separately or combined prior and the combined stream may be introduced into the aromatics complex. In the aromatics complex, the stream or streams can be processed to provide a recycled content paraxylene (r-paraxylene) stream. The r-paraxylene stream, which comprises recycled content paraxylene (r-pX), can also include non-recycled content hydrocarbon components, including non-recycled content paraxylene (pX). The r-paraxylene stream can include at least 55, at least 60, at least 65, at least 70, at least 75, at least 80, at least 85, at least 90, at least 95, at least 97, or at least 99 percent of r-pX, based on the total amount of r-pX and pX in the stream. The total amount of paraxylene in the r-paraxylene stream (including both pX and r-pX) can be at least 85, at least 90, at least 92, at least 95, at least 97, at least 99, or at least 99.5 weight percent. In some cases, all of the paraxylene in the r-paraxylene stream can be r-pX.

Referring now to FIG. 6, a schematic diagram of the main steps/zones of an aromatics complex as shown in FIG. 2 is provided. In one embodiment or in combination with any embodiment mentioned herein, a recycled content aromatics feed (r-aromatics feed) stream comprising predominantly C6 to C10 aromatics may be introduced into a first separation zone of the aromatics complex. The r-aromatics feed stream may comprise recycled content and it may also include non-recycled content. The stream may comprise at least 55, at least 60, at least 65, at least 70, at least 75, at least 80, or at least 85 weight percent of C6 to C10 aromatics.

The r-aromatics feed stream can comprise r-pyrolysis gasoline from one or more steam cracking facilities and/or r-reformate from one or more reformer units. At least a portion of the recycled content in these streams can be derived from waste plastic through processing one or more recycled content hydrocarbon streams such as r-pyoil, r-pygas, r-naphtha, r-light gas, or other streams in at least one steam cracking facility and/or at least one reformer unit of a refinery, according to one or more embodiments as described in further detail herein. Additionally, or in the alternative, a stream of aromatics (and/or recycled content aromatics, or r-aromatics) from one or more other processing facilities may also be included in the r-aromatics feed stream.

In one embodiment or in combination with any embodiment mentioned herein, the r-aromatics feed stream introduced into the aromatics complex (or one or more streams that make up this r-aromatics feed stream) can have one or more of the following properties (i) through (viii): (i) the stream(s) can comprise predominantly C6 to C10 (or C6 to C9) aromatics, or it can include at least 25, at least 35, at least 45, at least 55, at least 60, at least 65, at least 70, at least 75, at least 80, at least 85, or at least 90 weight percent of C6 to C10 (or C6 to C9) aromatic components; (ii), the stream(s) can comprise less than 75, less than 65, less than 55, less than 45, less than 35, less than 25, less than 15, or less than 10 weight percent of non-aromatic components; (iii) the streams can comprise at least 1, at least 2, at least 3, at least 5, or at least 10 and/or not more than 30, not more than 25, not more than 20, not more than 15, not more than 10, or not more than 7 weight percent of benzene, which may include recycled content benzene (r-benzene) and/or non-recycled content benzene; (iv) the stream(s) can comprise at least 5, at least 10, at least 15, or at least 20 and/or not more than 40, not more than 35, not more than 30, not more than 25, or not more than 20 weight percent of toluene, which can include both recycled content toluene (r-toluene) and/or non-recycled content toluene; (v) the stream(s) can comprise at least 2, at least 5, at least 10, at least 15, at least 20, or at least 25 weight percent and/or not more than 75, not more than 70, not more than 65, not more than 60, not more than 55, not more than 50, not more than 45, not more than 40, not more than 35, not more than 30, or not more than 25 weight percent of one or more of C8 aromatics (or recycled content C8 aromatics, r-C8 aromatics), C9 aromatics (or recycled content C9 aromatics, r-C9 aromatics), and C10 aromatics (or recycled content C10 aromatics, r-C10 aromatics, individually or in combination; (vi) the stream(s) can comprise at least 5, at least 10, or at least 15 and/or not more than 50, not more than 45, or not more than 40 weight percent of mixed xylenes, including recycled and non-recycled content xylenes; (vii) the stream(s) may comprise not more than 15, not more than 10, not more than 5, not more than 2, or not more than 1 weight percent of C5 and lighter components and/or C11 and heavier components; and (viii) the stream(s) can comprise a total amount of C6 to C10 (or C9 to C10) hydrocarbon components of at least 55, at least 60, at least 65, at least 70, at least 75, at least 80, at least 85, or at least 90 weight percent, based on the total weight of the stream Examples of C8 aromatics include, but are not limited to, mixed xylenes such as ortho-xylene, paraxylene, and meta-xylene, as well as ethylbenzene, and styrene, while C9 aromatics can include, for example, isopropyl benzene, propyl benzene, isomers of methyl ethyl benzene, isomers of methyl styrenes, and isomers of trimethyl benzene. Examples of C10 aromatics can include, but are not limited to, isomers of butyl benzene, isomers of diethyl benzene, and isomers of dimethyl ethyl benzene. One or more of these components, when present in the aromatics stream, may include recycled content and/or may include non-recycled content.

In one embodiment or in combination with any embodiment mentioned herein, the r-aromatics stream may comprise 20 to 80, or 25 to 75, or 30 to 60 weight percent benzene and/or 0.5 to 40, or 1 to 35, or 2 to 30 weight percent toluene, and/or 0.05 to 30, or 0.10 to 25, or 0.20 to 20 weight percent of C8 aromatics, based on the total weight of aromatics in the r-aromatics stream.

As shown in FIGS. 1 and 6, at least a portion of the r-aromatics stream (which may include, for example, an r-pyrolysis gasoline stream from a steam cracking facility and/or an r-reformate stream from a reformer unit of a refinery) can be introduced into an initial separation zone in the aromatics complex. In one embodiment or in combination with any embodiments mentioned herein, two or more of the feed streams (e.g., r-reformate, r-pyrolysis gasoline, r-aromatics, and yet-to-be-discussed r-raffinate) may also be introduced into the initial separation zone separately or two or more of these streams may be combined and the combined stream introduced into the separation zone.

As shown in FIG. 6, at least a portion of the r-aromatics feed stream may optionally be hydrotreated prior to entering the initial separation zone of the aromatics complex. When present, this hydrotreating zone may hydrogenate the streams to reduce at least a portion of the unsaturated carbon-carbon bonds to form saturated carbon-carbon bonds. The hydrotreating unit may include one or more hydrotreating (e.g., hydrogenation) reactors containing a catalyst, such as nickel, palladium, rhodium, ruthenium, or platinum-containing catalysts. The resulting hydrotreated (e.g., hydrogenated) stream may then be introduced into the initial separation zone of the aromatics complex, as shown in FIG. 6.

The initial separation zone of the aromatics complex shown in FIG. 6 can utilize any suitable method for separating at least a portion of the aromatics out of the feed streams introduced into the separation zone. In one embodiment or in combination with any embodiments mentioned herein, the initial separation zone may remove at least 50, at least 60, at least 75, at least 80, or at least 90 weight percent of the total amount of aromatics introduced into the separation zone, resulting in an aromatics-enriched predominantly benzene, toluene, and xylene (BTX) stream and an aromatics-depleted raffinate stream. The BTX stream may comprise at least at least 55, at least 65, at least 75, at least 85, or at least 90 weight percent C6 to C9 aromatics, while the raffinate stream can comprise less than 50, less than 40, less than 30, less than 20, or less than 10 weight percent C6 to C9 aromatics. When one or more of the feed streams to the initial separation zone comprise recycled content, the BTX stream can be a recycled content BTX (r-BTX) stream, and the raffinate stream may be a recycled content raffinate (r-raffinate) stream.

In addition to BTX, the r-BTX stream may include other aromatic and non-aromatic components. For example, the r-BTX (or BTX) stream may include at least 1, at least 2, at least 5, or at least 10 weight percent and/or not more than 25, not more than 20, not more than 15, or not more than 10 weight percent of C9 and heavier (or C10 and heavier) components. Such components can include C9 and heavier (or C10 and heavier) aromatic components as well as non-aromatic C9 and heavier (or C10 and heavier) components.

The separation step carried out in the initial separation zone of the aromatics complex can may be performed using any suitable type of separation, including extraction, distillation, and extractive distillation. When the separation step includes extraction or extractive distillation, it may utilize at least one solvent selected from the group consisting of sulfolane, furfural, tetraethylene glycol, dimethylsulfoxide, N, N-dimethylformamide, and N-methyl-2-pyrrolidone. When the initial separation step includes distillation, it may be performed in one or more distillation columns. Upon separation, the r-raffinate stream depleted in aromatics can be withdrawn from the separation step/zone. The r-raffinate stream comprises predominantly C5 and heavier, or C5 to C12 components, and may include not more than 20, not more than 15, not more than 10, not more than 5 weight, not more than 2, or not more than 1 percent of C6 to C10, or C6 to C9, or C6 to C8 aromatics (e.g., benzene, toluene, and xylenes). The r-raffinate stream withdrawn from the aromatics complex can comprise predominantly C4 to C8, C5 to C7, or C5 and C6 hydrocarbon components, or it can include at least 55, at least 60, at least 65, at least 70, or at least 75 weight percent of these compounds.

As shown in FIG. 2, the r-raffinate stream from the aromatics complex may optionally be introduced into a steam cracking facility and/or a reformer. Within the reformer and/or steam cracker, the r-raffinate stream may be further processed to form another r-pyrolysis gasoline and/or another r-reformate stream to provide another C6 to C10 aromatics (or r-C6 to C10 aromatics) stream that can be reintroduced into the aromatics complex.

Referring again to FIG. 6, a stream concentrated in recycled content benzene, toluene, and xylenes (r-BTX) can also be withdrawn from the initial separation step. This r-BTX stream comprises predominantly BTX and may include at least 60, at least 70, at least 80, at least 85, at least 90, or at least 95 BTX, including both recycled content BTX (r-BTX) and non-recycled content BTX, as applicable. The r-BTX stream can be introduced into a downstream BTX recovery zone, which utilizes one or more separation steps to provide streams concentrated in recycled content benzene (r-benzene), recycled content mixed xylenes (r-mixed xylenes), and recycled content toluene (r-toluene). Such separations can be performed according to any suitable method, including, for example, with one or more distillation columns or other separation equipment or steps such extraction, crystallization, and/or adsorption. As discussed previously, this r-BTX stream may include other C8 aromatics (such as ethylbenzene), as well as C9 and heavier (or C10 and heavier) components in addition to the benzene, toluene, and mixed xylenes. Components other than BTX in the r-BTX stream may be present in an amount of at least 1, at least 2, at least 5, or at least 10 weight percent and/or not more than 25, not more than 20, not more than 15, or not more than 10 weight percent.

As shown in FIG. 6, the r-benzene formed in BTX recovery step can be removed as a product stream from aromatics complex, while the r-mixed xylenes can be introduced into a second separation step for separating out recycled content ortho-xylene (r-oX), recycled content meta-xylene (r-mX), and/or recycled content paraxylene (r-pX) from the other components in the stream. In addition to comprising at least 25, at least 30, at least 35, at least 40, or at least 45 weight percent and/or not more than 70, not more than 65, not more than 60, or not more than 55 weight percent mixed xylenes, this stream of r-mixed xylenes may also include other C8 aromatics (such as ethylbenzene), as well as C9 and heavier (or C10 and heavier) aromatic and non-aromatic components. Such components (which may include recycled content or non-recycled content components) can be present in the r-BTX stream in an amount of at least 1, at least 2, at least 5, or at least 10 weight percent and/or not more than 35, not more than 30, not more than 25, not more than 20, not more than 15, not more than 10, or not more than 5 weight percent.

This second separation step can utilize one or more of distillation, extraction, crystallization, and adsorption to provide recycle content aromatics streams. For example, as shown in FIG. 6, the separation step can provide at least one of a recycled content paraxylene (r-paraxylene) stream, a recycled content metaxylene (r-metaxylene) stream, and a recycled content orthoxylene (r-orthoxylene) stream. Each of these streams may include both recycled and non-recycled content and can individually include at least 75, at least 80, at least 85, at least 90, at least 95, or at least 97 weight percent of paraxylene (r-pX and pX), metaxylene (r-mX and mX), or orthoxylene (r-oX and oX), respectively.

Additionally, at least a portion of the oX (or r-oX) and/or mX (or r-mX) can be subjected to isomerization to provide additional pX (or r-pX). After the isomerization, additional separation steps may be performed to provide individual streams of oX (or r-oX), mX (or r-mX), and pX (or r-pX).

As shown in FIG. 6, a stream of recycled content C9 and heavier components (r-C9+components) may also be withdrawn from the second separation step and all or a portion may be introduced into a transalkylation/disproportionation step along with a stream of r-toluene withdrawn from the BTX recovery step/zone. In the transalkylation/disproportionation step/zone, at least a portion of the toluene (or r-toluene) can be reacted in the presence of a regenerable fixed bed silica-alumina catalyst to provide mixed xylenes (or r-mixed xylenes) and benzene (or r-benzene). Alternatively, or in addition, at least a portion of the r-toluene can be reacted with methanol (and, optionally, recycled content methanol from waste plastic or sustainable content methanol from biomass) to provide recycled content paraxylene (r-paraxylene), which may be further processed as described herein. In some cases, this reaction may be performed within the aromatics complex over an acidic catalyst, preferably on a shape-selective molecular sieve catalyst such as ZSM-5, and the resulting r-paraxylene may be combined with other paraxylene (or r-paraxylene) recovered in the aromatics complex. As shown in FIG. 6, the benzene (or r-benzene) can be recovered as a product, while the r-mixed xylenes can be introduced into the second separation step/zone for further separation into a r-paraxylene stream, an r-orthoxylene stream, and a r-metaxylene stream.

Turning back to FIG. 2, at least a portion of the r-paraxylene stream withdrawn from the aromatics complex can be sent to a TPA production facility. In the TPA production facility, at least a portion of the pX (and/or r-pX) in the r-paraxylene stream can be oxidized in the presence of a solvent (e.g., acetic acid) and a catalyst to form recycled content crude terephthalic acid (r-CTA).

Thereafter, depending on the specific TPA production process utilized within the production facility, the r-CTA can either be oxidized again in a secondary or post-oxidation step or it can be hydrogenated in a treatment step to form recycled content purified terephthalic acid (r-PTA). All or a portion of the solvent may be removed from the r-CTA and swapped out for new solvent, which may be the same as or different than the original solvent. The resulting r-PTA slurry can be processed by, for example, drying, crystallization, and filtration to provide the final r-TPA product.

In one embodiment or in combination with any embodiments mentioned herein, as shown in FIG. 2, at least a portion of the r-TPA product can be introduced into a PET production facility and reacted with at least one diol (such as, for example, ethylene glycol) to form recycled content polyethylene terephthalate (r-PET). In one embodiment or in combination with any embodiments mentioned herein, the r-TPA and ethylene glycol (or, recycled content ethylene glycol, r-EG) can be polymerized in the presence of one or more comonomers, such as isophthalic acid or neopentyl glycol or cyclohexanedimethanol, to form a recycled content PET copolymer (r-co-PET).

### Definitions

It should be understood that the following is not intended to be an exclusive list of defined terms. Other definitions may be provided in the foregoing description, such as, for example, when accompanying the use of a defined term in context.

As used here, the term “light gas” refers to a hydrocarbon-containing stream comprising at least 50 weight percent of C4 and lighter hydrocarbon components. Light hydrocarbon gas may include other components such as nitrogen, carbon dioxide, carbon monoxide, and hydrogen, but these are typically present in amount of less than 20, less than 15, less than 10, or less than 5 weight percent, based on the total weight of the stream.

As used herein, the terms “median boiling point” or “T50” refers to the median boiling point of a process stream (i.e., the temperature value where 50 weight percent of the stream composition boils above the temperature value and 50 weight percent of the stream composition boils below the temperature value).

As used herein, the term “boiling point range” or “cut point” refers to the range of temperatures over which a specified petroleum fraction boils. The lower value in a boiling point range is the initial boiling point (IBP) temperature for that specified fraction and the higher value is the end point (EP) temperature for that specified fraction.

As used herein, the term “naphtha” refers to a physical mixture of hydrocarbon components separated in at least one distillation column of a refining facility that has a boiling point range between 90 to 380 °F.

As used herein, the term “light naphtha” refers to a specific portion of a naphtha cut in a refinery having a boiling point range between 90 and less than 230 °F.

As used herein, the term “heavy naphtha” refers to a specific portion of a naphtha cut in a refinery having a boiling point range between 230 and 380 °F.

As used herein, the terms “distillate” and “kerosine” refer to a physical mixture of hydrocarbon components separated in at least one distillation column of a refining facility that has a boiling point range greater than 380 to 520 °F.

As used herein, the term “hydrocracker distillate” refers to a distillate cut removed from a hydrocracker unit.

As used herein, the term “gas oil” refers to a physical mixture of hydrocarbon components separated in at least one distillation column of a refining facility that has a boiling point range greater than 520 to 1050 °F.

As used herein, the term “atmospheric gas oil” refers to a gas oil produced by the atmospheric distillation unit.

As used herein, the term “light gas oil” or “LGO” refers to a specific portion of gas oil cut in a refinery having a boiling point range between greater than 520 and 610 °F.

As used herein, “light vacuum gas oil” or “LVGO” refers to a light gas oil produced by the vacuum distillation unit.

As used herein, “light vacuum gas oil” or “LCGO” refers to a light gas oil produced by the coker unit.

As used herein, the term “heavy gas oil” or “HGO” refers to a specific portion of a gas oil cut in a refinery having a boiling point range between greater than 610 and 800 °F.

As used herein, “heavy vacuum gas oil” or “HVGO” refers to a heavy gas oil produced by the vacuum distillation unit.

As used herein, “heavy coker gas oil” or “HCGO” refers to a heavy gas oil produced by the coker unit.

As used herein, the term “vacuum gas oil” or “VGO” refers to a specific portion of a gas oil cut in a refinery having a boiling point range between greater than 800 and 1050 °F. Vacuum gas oil is separated from the initial crude oil using a vacuum distillation column operated at a pressure below atmospheric pressure.

As used herein, the term “residue” or “resid” refers to the heaviest cut from a distillation column in a refinery and having a boiling point range between greater than 1050 °F.

As used herein, the term “vacuum resid” refers to a resid product from the vacuum distillation column.

As used herein, the term “atmospheric resid” refers to a resid product from the atmospheric distillation column.

As used herein, the term “gas plant” refers to equipment, including one or more distillation columns as well as ancillary equipment as well as pumps, compressors, valves, etc. in a refinery for processing a hydrocarbon feed stream comprising predominantly C6 and lighter components to provide one or more purified streams of C1 to C6 alkanes and/or olefins.

As used herein, the term “saturated gas plant” refers to a gas plant in a refinery for processing a hydrocarbon feed stream comprising predominantly saturated hydrocarbons (alkanes). The feed stream to a saturated gas plant includes less than 5 weight percent of olefins, based on the total feed to the plant. The feed to a saturated gas plant in a refinery may come, directly or indirectly, from the crude distillation unit or vacuum distillation unit and may undergo little or no cracking.

As used herein, the term “unsaturated gas plant” refers to a gas plant in a refinery for processing a hydrocarbon feed stream comprising saturated hydrocarbons (alkanes) and unsaturated hydrocarbons (olefins). The feed stream to an unsaturated gas plant includes at least 5 weight percent of olefins, based on the total feed to the plant. The feed to a saturated gas plant in a refinery may come indirectly from the crude unit or vacuum distillation unit and may undergo one or more cracking steps prior to entering the gas plant.

As used herein, the term “gas oil cracker” refers to a cracking unit for processing a feed stream comprising predominantly gas oil and heavier components. Although a gas oil cracker can process lighter components, such as distillate and naphtha, at least 50 weight percent of the total feed to a gas oil cracker includes gas oil and heavier components. Gas oil crackers may be operated at temperatures of at least 350 °F, at least 400 °F, at least 450 °F, at least 500 °F, at least 550 °F, or at least 600 °F and/or not more than 1200 °F, not more than 1150 °C, not more than 1100 °F, not more than 1050 °F, not more than 1000 °F, not more than 900 °F, or not more than 800 °F. Gas oil crackers may be operated at or near atmospheric pressure (e.g., at a pressure of less than 5 psig, less than 2 psig, or 1 psig) or may be operated at elevated pressure (e.g., at a pressure of at least 5 psig, at least 10 psig, at least 25 psig, at least 50 psig, at least 100 psig, at least 250 psig, at least 500 psig, or at least 750 psig.) Additionally, the cracking in gas oil crackers may be carried with or without a catalyst, and it may or may not be conducted in the presence of hydrogen and/or steam.

As used herein, the term “fluidized catalytic cracker” or “FCC” refers to a set of equipment, including a reactor, a regenerator, a main fractionator, as well as ancillary equipment such as pipes, valves, compressors, and pumps, for reducing the molecular weight of a heavy hydrocarbon stream via catalytic cracking in a fluidized catalyst bed.

As used herein, the terms “reformer” or “catalytic reformer” refer to a process or facility in which a feedstock comprising predominantly C6-C10 alkanes is converted to a reformate comprising branched hydrocarbons and/or cyclic hydrocarbons in the presence of a catalyst.

As used herein, the term “reformate” refers to a liquid product stream produced by a catalytic reformer process.

As used herein, the term “hydroprocessing” refers to chemical processing of a hydrocarbon stream with or in the presence of hydrogen. Hydroprocessing is typically a catalytic process and includes hydrocracking and hydrotreating.

As used herein, the term “hydrocracking” refers a type of hydroprocessing where the hydrocarbon molecules are cracked (i.e., undergo a reduction in molecular weight).

As used herein, the term “hydrotreating” refers to a type of hydroprocessing that does not crack the hydrocarbon molecules, but instead removes oxygen, sulfur, and other heteroatoms by hydrogenolysis or to saturate unsaturated bonds by hydrogenation. It may or may not be carried out in the presence of a catalyst.

As used herein, the term “distillation” refers to separation of a mixture of components by boiling point difference.

As used herein, the term “atmospheric distillation” refers to distillation performed at a pressure at or near atmospheric, usually to separate crude oil and/or other streams into specified fractions for further processing.

As used herein, the term “vacuum distillation” refers to distillation performed at a pressure below atmospheric and, usually, at a pressure of less than 100 mm Hg at the top of the column.

As used herein, the term “coking” refers to thermal cracking of heavy hydrocarbons (usually atmospheric or vacuum column bottoms) performed to recover light, more valuable products such as naphtha, distillate, gas oil, and light gas.

As used herein, the term “aromatics complex” refers to a process or facility in which a mixed hydrocarbon feedstock, such as a reformate, is converted into one or more benzene, toluene, and/or xylene (BTX) product streams, such as a paraxylene product stream. The aromatics complex may comprise one or more processing steps, in which one or more components of the reformate are subjected to at least one of a separation step, a transalkylating step, a toluene disproportionation step, and/or an isomerization step. The separation step can comprise one or more of an extraction step, a distillation step, a crystallization step, and/or an adsorption step.

As used herein, the term “raffinate” refers to the aromatics-depleted stream removed from the initial separation step in the aromatics complex. Although most commonly used to refer to a stream withdrawn from an extraction step, the term “raffinate” as used with respect to the aromatics complex can also refer to a stream withdrawn from another type of separation, including, but not limited to, distillation or extractive distillation.

As used herein, the terms “pyrolysis oil” or “pyoil” refers to a composition obtained from pyrolysis that is liquid at 25 °C and 1 atm, absolute.

As used herein, the terms “pyrolysis gas” and “pygas” refer to a composition obtained from pyrolysis that is gaseous at 25 °C and 1 atm, absolute.

As used herein, the term “pyrolysis” refers to thermal decomposition of one or more organic materials at elevated temperatures in an inert (i.e., substantially oxygen free) atmosphere.

As used herein, the term “pyrolysis vapor” refers to the overhead or vapor-phase stream withdrawn from the separator in a pyrolysis facility used to remove r-pyrolysis residue from the r-pyrolysis effluent.

As used herein, the term “pyrolysis effluent” refers to the outlet stream withdrawn from the pyrolysis reactor in a pyrolysis facility.

As used herein, the term “r-pyrolysis residue” refers to a composition obtained from waste plastic pyrolysis that comprises predominantly pyrolysis char and pyrolysis heavy waxes.

As used herein, the term “pyrolysis char” refers to a carbon-containing composition obtained from pyrolysis that is solid at 200 °C and 1 atm, absolute.

As used herein, the term “pyrolysis heavy waxes” refers to C20+hydrocarbons obtained from pyrolysis that are not pyrolysis char, pyrolysis gas, or pyrolysis oil.

As used herein, the term “pyrolysis gasoline” refers to a hydrocarbon stream of predominantly C5 and heavier components removed from a quench section of a steam cracking facility. Typically, pyrolysis gasoline includes at least 10 weight percent of C6 to C9 aromatics.

As used herein, the term “lighter” refers to a hydrocarbon component or fraction having a lower boiling point than another hydrocarbon component or fraction.

As used herein, the term “heavier” refers to a hydrocarbon component or fraction having a higher boiling point than another hydrocarbon component or fraction.

As used herein, the term “upstream” refers to an item of facility that is positioned prior to another item or facility in a given process flow and may include intervening items and/or facilities.

As used herein, the term “downstream” refers to an item or facility that is positioned after another item or facility in a given process flow and may include intervening items and/or facilities.

As used herein, the term “alkane” refers to a saturated hydrocarbon including no carbon-carbon double bonds.

As used herein, the term “olefin” refers to an at least partially unsaturated hydrocarbon including at least one carbon-carbon double bond.

As used herein, the terms “Cx” or “Cx hydrocarbon” or “Cx component” refers to a hydrocarbon compound including “x” total carbons per molecule, and encompasses all olefins, paraffins, aromatics, heterocyclic, and isomers having that number of carbon atoms. For example, each of normal, iso, and tert-butane and butene and butadiene molecules would fall under the general description “C4” or “C4 components.”

As used herein, the terms “r-paraxylene” or “r-pX” refer to being or comprising a paraxylene product that is directly and/or indirectly derived from waste plastic.

As used herein, the term “cracking” refers to breaking down complex organic molecules into simpler molecules by the breaking of carbon-carbon bonds.

As used herein, the term “steam cracking” refers to thermal cracking of hydrocarbons in the presence of steam, usually performed in a furnace of the steam cracking facility.

As used herein, the term “co-located” refers to the characteristic of at least two objects being situated on a common physical site, and/or within five miles of each other, measured as a straight-line distance between two designated points.

As used herein, the term “commercial scale facility” refers to a facility having an average annual feed rate of at least 500 pounds per hour, averaged over one year.

As used herein, the terms “crude” and “crude oil” refer to a mixture of hydrocarbons that exists in liquid phase and is derived from natural underground reservoirs.

As used herein, the terms “recycle content” and “r-content” refer to being or comprising a composition that is directly and/or indirectly derived from waste plastic.

As used herein, the term “predominantly” means more than 50 percent by weight. For example, a predominantly propane stream, composition, feedstock, or product is a stream, composition, feedstock, or product that contains more than 50 weight percent propane.

As used herein, the term “waste material” refers to used, scrap, and/or discarded material.

As used herein, the terms “waste plastic” and “plastic waste” refer to used, scrap, and/or discarded plastic materials.

As used herein, the terms “mixed plastic waste” and “MPW” refer to a mixture of at least two types of waste plastics including, but not limited to the following plastic types: polyethylene terephthalate (PET), one or more polyolefins (PO), and polyvinylchloride (PVC).

As used herein, the term “fluid communication” refers to the direct or indirect fluid connection between two or more processing, storage, or transportation facilities or zones.

As used herein, the terms “a,” “an,” and “the” mean one or more.

As used herein, the term “and/or,” when used in a list of two or more items, means that any one of the listed items can be employed by itself or any combination of two or more of the listed items can be employed. For example, if a composition is described as containing components A, B, and/or C, the composition can contain A alone; B alone; C alone; A and B in combination; A and C in combination, B and C in combination; or A, B, and C in combination.

As used herein, the phrase “at least a portion” includes at least a portion and up to and including the entire amount or time period.

As used herein, the term “chemical recycling” refers to a waste plastic recycling process that includes a step of chemically converting waste plastic polymers into lower molecular weight polymers, oligomers, monomers, and/or non-polymeric molecules (e.g., hydrogen, carbon monoxide, methane, ethane, propane, ethylene, and propylene) that are useful by themselves and/or are useful as feedstocks to another chemical production process(es).

As used herein, the terms “mechanical recycling” and “physical recycling” refer to a recycling process that includes a step of melting waste plastic and forming the molten plastic into a new intermediate product (e.g., pellets or sheets) and/or a new end product (e.g., bottles).

As used herein, the terms “comprising,” “comprises,” and “comprise” are open-ended transition terms used to transition from a subject recited before the term to one or more elements recited after the term, where the element or elements listed after the transition term are not necessarily the only elements that make up the subject.

As used herein, the term “cracking” refers to breaking down complex organic molecules into simpler molecules by the breaking of carbon-carbon bonds.

As used herein, the terms “including,” “include,” and “included” have the same open-ended meaning as “comprising,” “comprises,” and “comprise” provided above.

As used herein, the term “predominantly” means more than 50 percent by weight. For example, a predominantly propane stream, composition, feedstock, or product is a stream, composition, feedstock, or product that contains more than 50 weight percent propane.

As used herein, the term “hydrocarbon” refers to an organic chemical compound that includes only carbon and hydrogen atoms.

As used herein, the term “organic chemical compound,” refers to a chemical compound that includes carbon and hydrogen atoms, but also includes oxygen and/or nitrogen atoms.

As used herein, the term “chemical pathway” refers to the chemical processing step or steps (e.g., chemical reactions, physical separations, etc.) between an input material and a product, where the input material is used to make the product.

As used herein, the terms “credit-based recycled content,” “non-physical recycled content,” and “indirect recycled content” all refer to matter that is not physically traceable back to a waste material, but to which a recycled content credit has been attributed.

As used herein, the term “directly derived” refers to having at least one physical component originating from waste material.

As used herein, the term “indirectly derived” refers to having an applied recycled content (i) that is attributable to waste material, but (ii) that is not based on having a physical component originating from waste material.

As used herein, the term “located remotely” refers to a distance of at least 0.1, 0.5, 1, 5, 10, 50, 100, 500, or 1000 miles between two facilities, sites, or reactors.

As used herein, the term “mass balance” refers to a method of tracing recycled content based on the mass of the recycled content in a product.

As used herein, the terms “physical recycled content” and “direct recycled content” both refer to matter physically present in a product and that is physically traceable back to a waste material.

As used herein, the term “recycled content” refers to being or comprising a composition that is directly and/or indirectly derived from recycle waste material. Recycled content is used generically to refer to both physical recycled content and credit-based recycled content. Recycled content is also used as an adjective to describe a product having physical recycled content and/or credit-based recycled content.

As used herein, the term “recycled content credit” refers to a non-physical measure of recycled content obtained from a mass of waste plastic that can be directly or indirectly (i.e., via a digital inventory) attributed to a product second material.

As used herein, the term “total recycled content” refers to the cumulative amount of physical recycled content and credit-based recycled content from all sources.

As used herein, the term “waste material” refers to used, scrap, and/or discarded material.

As used herein, the terms “waste plastic” and “plastic waste” refer to used, scrap, and/or discarded plastic materials, including post industrial or pre-consumer waste plastic and post-consumer waste plastic.

As used herein, the term “hydroprocessing unit” refers to a set of equipment, including reaction vessels, a drier, and a main fractionator, as well as ancillary equipment such as pipes, valves, compressors, and pumps, for chemically processing a hydrocarbon stream in the presence of hydrogen. Specific examples of hydroprocessing units include a hydrocracker (or hydrocracking unit) configured to carry out a hydrocracking process and a hydrotreater (or hydrotreating unit) configured to carry out a hydrotreating process.

As used herein, the term “coker” or “coking unit” refers to a set of equipment, including reaction vessels, a drier, and a main fractionator, as well as ancillary equipment such as pipes, valves, compressors, and pumps, for reducing the molecular weight of a heavy hydrocarbon stream via thermal cracking or coking.

As used herein, the terms “steam cracking facility” or “steam cracker” refer to all of the equipment needed to carry out the processing steps for thermally cracking a hydrocarbon feed stream in the presence of steam to form one or more cracked hydrocarbon products. Examples include, but are not limited to, olefins such as ethylene and propylene. The facility may include, for example, a steam cracking furnace, cooling equipment, compression equipment, separation equipment, as well as the pipes, valves, tanks, pumps, etc. needed to carry out the processing steps.

As used herein, the terms “refinery,” “refining facility,” and “petroleum refinery,” refer to all of the equipment needed to carry out the processing steps for separating and converting petroleum crude oil into multiple hydrocarbon fractions, one or more of which can be used as a fuel source, lube oil, bitumen, coke, and as an intermediate for other chemical products.” The facility may include, for example, separation equipment, thermal or catalytic cracking equipment, chemical reactors, and product blending equipment, as well as the pipes, valves, tanks, pumps, etc. needed to carry out the processing steps.

As used herein, the term “pyrolysis facility,” refers to all of the equipment needed to carry out the processing steps for pyrolyzing a hydrocarbon-containing feed stream, which can include or be waste plastic. The facility may include, for example, reactors, cooling equipment, and separation equipment, as well as the pipes, valves, tanks, pumps, etc. needed to carry out the processing steps.

As used herein, the term “terephthalic acid production facility,” or “TPA production facility,” refers to all of the equipment needed to carry out the processing steps for forming terephthalic acid from paraxylene. The facility may include, for example, reactors, separators, cooling equipment, separation equipment such as filters or crystallizers, as well as the pipes, valves, tanks, pumps, etc. needed to carry out the processing steps.

As used herein, the term “polyethylene terephthalate production facility,” or “PET production facility,” refers to all of the equipment needed to carry out the processing steps for forming polyethylene terephthalate (PET) from a terephthalate, ethylene glycol, and, optionally, one or more additional monomers. The facility may include, for example, polymerization reactors, cooling equipment, and equipment to recover solidified and/or pelletized PET, as well as the pipes, valves, tanks, pumps, etc. needed to carry out the processing steps.

As used herein the term “chemical processing facility,” refers to all of the equipment needed to carry out the processing steps for one or more chemical processes for converting a starting material to a final chemical product. The facility may include, for example, separation or treatment equipment, reaction equipment, and equipment to recover a final product, as well as the pipes, valves, tanks, pumps, etc. needed to carry out the processing steps.

### Claims not Limited to Disclosed Embodiments

The preferred forms of the invention described above are to be used as illustration only and should not be used in a limiting sense to interpret the scope of the present invention. Modifications to the exemplary embodiments, set forth above, could be readily made by those skilled in the art without departing from the spirit of the present invention.

The inventors hereby state their intent to rely on the Doctrine of Equivalents to determine and assess the reasonably fair scope of the present invention as it pertains to any apparatus not materially departing from but outside the literal scope of the invention as set forth in the following claims.

1. A method for producing recycled content paraxylene (r-paraxylene), the method comprising:
(a) distilling recycled content pyrolysis oil (r-pyoil) and crude oil in at least one distillation column to produce at least one recycled content heavy hydrocarbon (r-heavy hydrocarbon) stream having a median boiling point (T50) of greater than 400 °F;
(b) reducing an average molecular weight of the r-heavy hydrocarbon stream to produce at least one recycled content naphtha (r-naphtha) stream, wherein the r-naphtha stream comprises at least one recycled content aromatics (r-aromatics) stream and/or wherein at least a portion of the r-naphtha stream is further processed to produce at least r-aromatics stream; and
(c) processing at least a portion of the r-aromatics stream within an aromatics complex to produce a recycled content paraxylene (r-paraxylene) stream comprising at least 85 weight percent paraxylene.

2. The method of claim 1, wherein at least a portion of the r-heavy hydrocarbon stream is hydrotreated before the reducing of (b).

3. The method of claim 1, wherein the r-heavy hydrocarbon stream is not hydrotreated before the reducing of step (b).

4. The method of claim 1, wherein the r-heavy hydrocarbon stream comprises one or more of:
(i) a recycled content atmospheric gas oil (r-AGO);
(ii) a recycled content atmospheric residue (r-atmospheric resid);
(iii) a recycled content vacuum gas oil (r-VGO); and/or
(iv) a recycled content vacuum residue (r-vacuum resid).

5. A method for producing recycled content paraxylene (r-paraxylene), the method comprising:
(a) distilling recycled content pyrolysis oil (r-pyoil) and crude oil in an atmospheric distillation column to produce at least one recycled content heavy hydrocarbon (r-heavy hydrocarbon) stream having a T50 of greater than 650 °F;
(b) separating at least a portion of the r-heavy hydrocarbon stream in a vacuum distillation unit to produce at least a recycled content light vacuum gas oil (r-LVGO) stream having a T50 of greater than 650 °F and not more than 800 °F;
(c) catalytically cracking at least a portion of the r-LVGO stream in a fluidized catalytic cracking (FCC) unit to produce at least one recycled content naphtha (r-naphtha) stream, wherein the r-naphtha stream comprises at least one recycled content aromatics (r-aromatics) stream and/or wherein at least a portion of the r-naphtha stream is further processed to produce an r-aromatics stream; and
(d) processing at least a portion of the r-aromatics stream within an aromatics complex to produce an r-paraxylene stream comprising at least 85 weight percent paraxylene.

6. The method of claim 5, wherein at least a portion of the r-LVGO stream is hydrotreated before the catalytically cracking of step (c).

7. The method of claim 5, wherein at least a portion of the r-LVGO stream is not hydrotreated before the cracking of step (c).

8. A method for producing recycled content paraxylene (r-pX), the method comprising:
(a) distilling recycled content pyrolysis oil (r-pyoil) and crude oil in an atmospheric distillation column to produce a recycled content atmospheric gas oil (r-AGO) stream having a T50 of greater than 400°F and not more than 650 °F;
(b) catalytically cracking at least a portion of the r-AGO stream in an FCC unit to produce at least one recycled content naphtha (r-naphtha) stream, wherein the r-naphtha stream comprises at least one recycled content aromatics (r-aromatics) stream and/or wherein at least a portion of the r-naphtha stream is further processed to produce at least one r-aromatics stream; and
(c) processing at least a portion of the r-aromatics stream within an aromatics complex to produce a recycled content paraxylene (r-paraxylene) stream comprising at least 85 weight percent paraxylene.

9. The method of claim 8, wherein at least a portion of the r-AGO stream is hydrotreated before the catalytically cracking of step (b).

10. The method of claim 8, wherein at least a portion of the r-AGO stream is not hydrotreated before the cracking of step (b).

11. A method for producing recycled content paraxylene (r-paraxylene), the method comprising:
(a) distilling recycled content pyrolysis oil (r-pyoil) and crude oil in an atmospheric distillation column to produce at least one recycled content heavy hydrocarbon (r-heavy hydrocarbon) stream having a median boiling point (T50) of greater than 650 °F and a recycled content atmospheric gas oil (r-AGO) stream having a T50 of greater than 400 °F and not more than 650 °F;
(b) separating at least a portion of the r-heavy hydrocarbon stream in a vacuum distillation unit (VDU) to produce at least a recycled content light vacuum gas oil (r-LVGO) stream having a T50 of greater than 650 °F and not more than 800 °F;
(c) catalytically cracking at least a portion of the r-AGO stream and at least a portion of the r-LVGO stream in a fluidized catalytic cracking (FCC) unit to produce at least one recycled content naphtha (r-naphtha) stream, wherein the r-naphtha stream comprises at least one recycled content aromatics (r-aromatics) stream and/or wherein at least a portion of the r-naphtha stream is further processed to produce at least one r-aromatics stream; and
(d) processing at least a portion of the r-aromatics stream within an aromatics complex to produce a recycled content paraxylene (r-paraxylene) stream comprising at least 85 weight percent paraxylene.

12. The method of claim 11, wherein at least a portion of the r-AGO stream and/or at least a portion of the r-LVGO stream is hydrotreated before the catalytically cracking of step (c).

13. The method of claim 11, wherein both of the r-AGO stream and the r-LVGO stream are not hydrotreated before the cracking of step (c).

14. A method for producing a recycled content organic chemical compound (r-organic chemical compound), the method comprising:
(a) introducing a recycled content aromatics (r-aromatics) stream into an aromatics complex, wherein the r-aromatics stream is obtained by distilling recycled content pyrolysis oil (r-pyoil) and crude oil in at least one distillation column of a refinery to produce at least one recycled content heavy hydrocarbon (r-heavy hydrocarbon) stream having a median boiling point (T50) of greater than 400 °F, reducing an average molecular weight of the r-heavy hydrocarbon stream to produce at least one recycled content naphtha (r-naphtha) stream, wherein the r-naphtha stream comprises the r-aromatics stream and/or wherein at least a portion of the r-naphtha stream is further processed to produce the r-aromatics stream; and
(b) processing at least a portion of the r-aromatics stream in the aromatics complex to provide a recycled content aromatics (r-aromatics) stream comprising at least 85 weight percent paraxylene.

15. A method for producing a recycled content organic chemical compound (r-organic chemical compound), the method comprising:
(a) introducing a stream of recycled content paraxylene (r-paraxylene) into a terephthalic acid (TPA) production facility, wherein at least a portion of the r-paraxylene is obtained by distilling recycled content pyrolysis oil (r-pyoil) and crude oil in at least one distillation column of a refinery to produce at least one recycled content heavy hydrocarbon (r-heavy hydrocarbon) stream having a median boiling point (T50) of greater than 400 °F, reducing an average molecular weight of at least a portion of the r-heavy hydrocarbon stream to produce at least one recycled content naphtha (r-naphtha) stream, wherein the r-naphtha stream comprises a recycled content aromatics (r-aromatics) stream and/or wherein at least a portion of the r-naphtha stream is further processed to produce the r-aromatics stream, and processing the r-aromatics stream in an aromatics complex to provide the r-paraxylene stream; and
(b) processing at least a portion of the r-paraxylene stream in the TPA production facility to provide recycled content purified terephthalic acid (r-PTA).

16. The method of claim 1, wherein at least a portion of the r-naphtha stream is hydrotreated before the processing of step (b).

17. The method of claim 1, wherein at least a portion of the r-naphtha stream is not hydrotreated before the processing of step (b).

18. The method of claim 1, wherein the r-naphtha stream comprises recycled content light naphtha (r-light naphtha) comprising predominantly C5 hydrocarbons and recycled content heavy naphtha (r-heavy naphtha) comprising predominantly C6 and heavier hydrocarbons.

19. The method of claim 18, wherein at least a portion of the r-heavy naphtha is:
(i) processed in the aromatics complex; and/or
(ii) catalytically reformed in a reformer unit to produce an aromatics-containing recycled content reformate (r-reformate) stream that is processed in the aromatics complex.

20. The method of claim 19, wherein at least a portion of the r-heavy naphtha is hydrotreated before being processed (i) and/or before being catalytically reformed (ii).
